SEQUENCE LISTING (314 sequences)

SEQ ID NO: 1 - DNA, 45 bp, Artificial Sequence
  Description of Artificial Sequence:variant 1 nuclear hormone receptor family zinc finger domain
tataatgcactgacctgtgaggggtgtaaaggtttcttcaggaga

SEQ ID NO: 2 - protein, 15 aa, Artificial Sequence
  Description of Artificial Sequence:variant 1 nuclear hormone receptor family zinc finger domain
YNALTCEGCKGFFRR

SEQ ID NO: 3 - DNA, 45 bp, Artificial Sequence
  Description of Artificial Sequence:variant 1 nuclear hormone receptor family zinc finger domain
tacggcgtgcgcacctgtgagggctgcaaaggcttctttaagcgc

SEQ ID NO: 4 - protein, 15 aa, Artificial Sequence
  Description of Artificial Sequence:variant 1 nuclear hormone receptor family zinc finger domain
YGVRTCEGCKGFFKR

SEQ ID NO: 5 - DNA, 45 bp, Artificial Sequence; identical to SEQ ID NO: 3
  Description of Artificial Sequence:variant 1 nuclear hormone receptor family zinc finger domain

SEQ ID NO: 6 - protein, 15 aa, Artificial Sequence
  Description of Artificial Sequence:variant 1 nuclear hormone receptor family zinc finger domain
YGVRTCEGCKGFFKR

SEQ ID NO: 7 - DNA, 45 bp, Artificial Sequence
  Description of Artificial Sequence:variant 1 nuclear hormone receptor family zinc finger domain
tacggcgtgcgaacctgcgagggctgcaagggctttttcaagaga

SEQ ID NO: 8 - protein, 15 aa, Artificial Sequence
  Description of Artificial Sequence:variant 1 nuclear hormone receptor family zinc finger domain
YGVRTCEGCKGFFKR

SEQ ID NO: 9 - DNA, 45 bp, Artificial Sequence
  Description of Artificial Sequence:variant 1 nuclear hormone receptor family zinc finger domain
tatggtgtccgcacatgtgagggctgcaagggcttcttcaagcgc

SEQ ID NO: 10 - protein, 15 aa, Artificial Sequence
  Description of Artificial Sequence:variant 1 nuclear hormone receptor family zinc finger domain
YGVRTCEGCKGFFKR

SEQ ID NO: 11 - DNA, 45 bp, Artificial Sequence
  Description of Artificial Sequence:variant 1 nuclear hormone receptor family zinc finger domain
tatggagcagtaacttgtgaaggctgcaaaggattttttaaaaga

SEQ ID NO: 12 - protein, 15 aa, Artificial Sequence
  Description of Artificial Sequence:variant 1 nuclear hormone receptor family zinc finger domain
YGAVTCEGCKGFFKR

SEQ ID NO: 13 - DNA, 45 bp, Artificial Sequence
  Description of Artificial Sequence:variant 1 nuclear hormone receptor family zinc finger domain
tacggggttatcacctgtgaggggtgcaagggcttcttccgccgg

SEQ ID NO: 14 - protein, 15 aa, Artificial Sequence
  Description of Artificial Sequence:variant 1 nuclear hormone receptor family zinc finger domain
YGVITCEGCKGFFRR

SEQ ID NO: 15 - DNA, 45 bp, Artificial Sequence
  Description of Artificial Sequence:variant 1 nuclear hormone receptor family zinc finger domain
tacggagtcatcacatgtgaaggctgcaagggattctttaggagg

SEQ ID NO: 16 - protein, 15 aa, Artificial Sequence
  Description of Artificial Sequence:variant 1 nuclear hormone receptor family zinc finger domain
YGVITCEGCKGFFRR

SEQ ID NO: 17 - DNA, 45 bp, Artificial Sequence
  Description of Artificial Sequence:variant 1 nuclear hormone receptor family zinc finger domain
tatggtgtcattacatgtgaaggctgcaagggctttttcaggaga

SEQ ID NO: 18 - protein, 15 aa, Artificial Sequence
  Description of Artificial Sequence:variant 1 nuclear hormone receptor family zinc finger domain
YGVITCEGCKGFFRR

SEQ ID NO: 19 - DNA, 45 bp, Artificial Sequence
  Description of Artificial Sequence:variant 2 nuclear hormone receptor family zinc finger domain
tatggagtgtacagctgcgaggggtgcaagggcttcttcaagcgg

SEQ ID NO: 20 - protein, 15 aa, Artificial Sequence
  Description of Artificial Sequence:variant 2 nuclear hormone receptor family zinc finger domain
YGVYSCEGCKGFFKR

SEQ ID NO: 21 - DNA, 45 bp, Artificial Sequence
  Description of Artificial Sequence:variant 2 nuclear hormone receptor family zinc finger domain
tacggggtttacagctgtgagggttgcaagggcttcttcaaacgc

SEQ ID NO: 22 - protein, 15 aa, Artificial Sequence
  Description of Artificial Sequence:variant 2 nuclear hormone receptor family zinc finger domain
YGVYSCEGCKGFFKR

SEQ ID NO: 23 - DNA, 45 bp, Artificial Sequence
  Description of Artificial Sequence:variant 2 nuclear hormone receptor family zinc finger domain
tacggggtatacagttgtgaaggctgcaaagggttcttcaagagg

SEQ ID NO: 24 - protein, 15 aa, Artificial Sequence
  Description of Artificial Sequence:variant 2 nuclear hormone receptor family zinc finger domain
YGVYSCEGCKGFFKR

SEQ ID NO: 25 - DNA, 45 bp, Artificial Sequence
  Description of Artificial Sequence:variant 2 nuclear hormone receptor family zinc finger domain
tacaacgtgctcagctgcgaaggctgcaagggcttcttccggcgc

SEQ ID NO: 26 - protein, 15 aa, Artificial Sequence
  Description of Artificial Sequence:variant 2 nuclear hormone receptor family zinc finger domain
YNVLSCEGCKGFFRR

SEQ ID NO: 27 - DNA, 45 bp, Artificial Sequence
  Description of Artificial Sequence:variant 2 nuclear hormone receptor family zinc finger domain
tacaatgttctgagctgcgagggctgcaagggattcttccgccgc

SEQ ID NO: 28 - protein, 15 aa, Artificial Sequence
  Description of Artificial Sequence:variant 2 nuclear hormone receptor family zinc finger domain
YNVLSCEGCKGFFRR

SEQ ID NO: 29 - DNA, 45 bp, Artificial Sequence
  Description of Artificial Sequence:variant 2 nuclear hormone receptor family zinc finger domain
tatgggatcatctcctgtgagggctgcaaagggtttttcaagcgg

SEQ ID NO: 30 - protein, 15 aa, Artificial Sequence
  Description of Artificial Sequence:variant 2 nuclear hormone receptor family zinc finger domain
YGIISCEGCKGFFKR

SEQ ID NO: 31 - DNA, 45 bp, Artificial Sequence
  Description of Artificial Sequence:variant 2 nuclear hormone receptor family zinc finger domain
tatggggtcagctcttgtgaaggctgcaagggcttctttcgccga

SEQ ID NO: 32 - protein, 15 aa, Artificial Sequence
  Description of Artificial Sequence:variant 2 nuclear hormone receptor family zinc finger domain
YGVSSCEGCKGFFRR

SEQ ID NO: 33 - DNA, 45 bp, Artificial Sequence; identical to SEQ ID NO: 31
  Description of Artificial Sequence:variant 2 nuclear hormone receptor family zinc finger domain

SEQ ID NO: 34 - protein, 15 aa, Artificial Sequence
  Description of Artificial Sequence:variant 2 nuclear hormone receptor family zinc finger domain
YGVSSCEGCKGFFRR

SEQ ID NO: 35 - DNA, 45 bp, Artificial Sequence
  Description of Artificial Sequence:variant 2 nuclear hormone receptor family zinc finger domain
tatggggctgtcagttgtgaaggttgcaaaggtttcttcaaaagg

SEQ ID NO: 36 - protein, 15 aa, Artificial Sequence
  Description of Artificial Sequence:variant 2 nuclear hormone receptor family zinc finger domain
YGAVSCEGCKGFFKR

SEQ ID NO: 37 - DNA, 45 bp, Artificial Sequence
  Description of Artificial Sequence:variant 3 nuclear hormone receptor family zinc finger domain
tacggtgtcttcacctgcgagggctgcaagagctttttcaagcga

SEQ ID NO: 38 - protein, 15 aa, Artificial Sequence
  Description of Artificial Sequence:variant 3 nuclear hormone receptor family zinc finger domain
YGVFTCEGCKSFFKR

SEQ ID NO: 39 - DNA, 45 bp, Artificial Sequence
  Description of Artificial Sequence:variant 3 nuclear hormone receptor family zinc finger domain
tacggccagttcacgtgcgagggctgcaagagcttcttcaagcgc

SEQ ID NO: 40 - protein, 15 aa, Artificial Sequence
  Description of Artificial Sequence:variant 3 nuclear hormone receptor family zinc finger domain
YGQFTCEGCKSFFKR

SEQ ID NO: 41 - DNA, 45 bp, Artificial Sequence
  Description of Artificial Sequence:variant 4 nuclear hormone receptor family zinc finger domain
tacggggtctacgcctgcgacggctgctcaggttttttcaaacgg

SEQ ID NO: 42 - protein, 15 aa, Artificial Sequence
  Description of Artificial Sequence:variant 4 nuclear hormone receptor family zinc finger domain
YGVYACDGCSGFFKR

SEQ ID NO: 43 - DNA, 45 bp, Artificial Sequence
  Description of Artificial Sequence:variant 4 nuclear hormone receptor family zinc finger domain
tatggcatctatgcctgcaacggctgcagcggcttcttcaagagg

SEQ ID NO: 44 - protein, 15 aa, Artificial Sequence
  Description of Artificial Sequence:variant 4 nuclear hormone receptor family zinc finger domain
YGIYACNGCSGFFKR

SEQ ID NO: 45 - DNA, 45 bp, Artificial Sequence
  Description of Artificial Sequence:variant 5 nuclear hormone receptor family zinc finger domain
tatggggcatccacctgtgatgggtgcaagggtttcttcagacgc

SEQ ID NO: 46 - protein, 15 aa, Artificial Sequence
  Description of Artificial Sequence:variant 5 nuclear hormone receptor family zinc finger domain
YGASTCDGCKGFFRR

SEQ ID NO: 47 - DNA, 45 bp, Artificial Sequence
  Description of Artificial Sequence:variant 5 nuclear hormone receptor family zinc finger domain
tacggtgcctcgagctgtgacggctgcaagggcttcttccggagg

SEQ ID NO: 48 - protein, 15 aa, Artificial Sequence
  Description of Artificial Sequence:variant 5 nuclear hormone receptor family zinc finger domain
YGASSCDGCKGFFRR

SEQ ID NO: 49 - DNA, 45 bp, Artificial Sequence
  Description of Artificial Sequence:variant 6 nuclear hormone receptor family zinc finger domain
tatggggtcagcgcctgtgagggctgcaagggcttcttccgccgc

SEQ ID NO: 50 - protein, 15 aa, Artificial Sequence
  Description of Artificial Sequence:variant 6 nuclear hormone receptor family zinc finger domain
YGVSACEGCKGFFRR

SEQ ID NO: 51 - DNA, 45 bp, Artificial Sequence
  Description of Artificial Sequence:variant 6 nuclear hormone receptor family zinc finger domain
tatggggtcagcgcctgtgagggatgtaagggctttttccgcaga

SEQ ID NO: 52 - protein, 15 aa, Artificial Sequence
  Description of Artificial Sequence:variant 6 nuclear hormone receptor family zinc finger domain
YGVSACEGCKGFFRR

SEQ ID NO: 53 - DNA, 45 bp, Artificial Sequence
  Description of Artificial Sequence:variant 6 nuclear hormone receptor family zinc finger domain
tacggtgtgcacgcctgcgagggctgcaagggctttttccgtcgg

SEQ ID NO: 54 - protein, 15 aa, Artificial Sequence
  Description of Artificial Sequence:variant 6 nuclear hormone receptor family zinc finger domain
YGVHACEGCKGFFRR

SEQ ID NO: 55 - DNA, 45 bp, Artificial Sequence
  Description of Artificial Sequence:variant 6 nuclear hormone receptor family zinc finger domain
tatggagttcatgcttgcgaaggctgtaagggtttctttcggaga

SEQ ID NO: 56 - protein, 15 aa, Artificial Sequence
  Description of Artificial Sequence:variant 6 nuclear hormone receptor family zinc finger domain
YGVHACEGCKGFFRR

SEQ ID NO: 57 - DNA, 45 bp, Artificial Sequence
  Description of Artificial Sequence:variant 6 nuclear hormone receptor family zinc finger domain
tacggtgttcatgcatgtgaggggtgcaagggcttcttccgtcgt

SEQ ID NO: 58 - protein, 15 aa, Artificial Sequence
  Description of Artificial Sequence:variant 6 nuclear hormone receptor family zinc finger domain
YGVHACEGCKGFFRR

SEQ ID NO: 59 - DNA, 45 bp, Artificial Sequence
  Description of Artificial Sequence:variant 6 nuclear hormone receptor family zinc finger domain
tacggagtccacgcgtgtgaaggctgcaagggcttctttcggcga

SEQ ID NO: 60 - protein, 15 aa, Artificial Sequence
  Description of Artificial Sequence:variant 6 nuclear hormone receptor family zinc finger domain
YGVHACEGCKGFFRR

SEQ ID NO: 61 - DNA, 45 bp, Artificial Sequence
  Description of Artificial Sequence:variant 6 nuclear hormone receptor family zinc finger domain
tatggagttcatgcttgtgaaggatgcaagggtttcttccggaga

SEQ ID NO: 62 - protein, 15 aa, Artificial Sequence
  Description of Artificial Sequence:variant 6 nuclear hormone receptor family zinc finger domain
YGVHACEGCKGFFRR

SEQ ID NO: 63 - DNA, 45 bp, Artificial Sequence
  Description of Artificial Sequence:variant 7 nuclear hormone receptor family zinc finger domain
ttcaatgtcatgacatgtgaaggatgcaagggctttttcaggagg

SEQ ID NO: 64 - protein, 15 aa, Artificial Sequence
  Description of Artificial Sequence:variant 7 nuclear hormone receptor family zinc finger domain
FNVMTCEGCKGFFRR

SEQ ID NO: 65 - DNA, 45 bp, Artificial Sequence
  Description of Artificial Sequence:variant 7 nuclear hormone receptor family zinc finger domain
tttaatgcgctgacttgtgagggctgcaagggtttcttcaggaga

SEQ ID NO: 66 - protein, 15 aa, Artificial Sequence
  Description of Artificial Sequence:variant 7 nuclear hormone receptor family zinc finger domain
FNALTCEGCKGFFRR

SEQ ID NO: 67 - DNA, 45 bp, Artificial Sequence
  Description of Artificial Sequence:variant 7 nuclear hormone receptor family zinc finger domain
taccgctgtatcacgtgtgaaggctgcaagggtttctttagaaga

SEQ ID NO: 68 - protein, 15 aa, Artificial Sequence
  Description of Artificial Sequence:variant 7 nuclear hormone receptor family zinc finger domain
YRCITCEGCKGFFRR

SEQ ID NO: 69 - DNA, 45 bp, Artificial Sequence
  Description of Artificial Sequence:variant 7 nuclear hormone receptor family zinc finger domain
taccgctgtatcacttgtgagggctgcaagggcttctttcgccgc

SEQ ID NO: 70 - protein, 15 aa, Artificial Sequence
  Description of Artificial Sequence:variant 7 nuclear hormone receptor family zinc finger domain
YRCITCEGCKGFFRR

SEQ ID NO: 71 - DNA, 45 bp, Artificial Sequence
  Description of Artificial Sequence:variant 7 nuclear hormone receptor family zinc finger domain
tacggactgctcacgtgtgagagctgcaagggcttcttcaagcgc

SEQ ID NO: 72 - protein, 15 aa, Artificial Sequence
  Description of Artificial Sequence:variant 7 nuclear hormone receptor family zinc finger domain
YGLLTCESCKGFFKR

SEQ ID NO: 73 - DNA, 45 bp, Artificial Sequence
  Description of Artificial Sequence:variant 7 nuclear hormone receptor family zinc finger domain
tatgggctcctcacctgtgaaagctgcaagggattttttaagcga

SEQ ID NO: 74 - protein, 15 aa, Artificial Sequence
  Description of Artificial Sequence:variant 7 nuclear hormone receptor family zinc finger domain
YGLLTCESCKGFFKR

SEQ ID NO: 75 - DNA, 45 bp, Artificial Sequence
  Description of Artificial Sequence:variant 8 nuclear hormone receptor family zinc finger domain
tatggggtagtcacctgtggcagctgcaaagttttcttcaaaaga

SEQ ID NO: 76 - protein, 15 aa, Artificial Sequence
  Description of Artificial Sequence:variant 8 nuclear hormone receptor family zinc finger domain
YGVVTCGSCKVFFKR

SEQ ID NO: 77 - DNA, 45 bp, Artificial Sequence
  Description of Artificial Sequence:variant 8 nuclear hormone receptor family zinc finger domain
tatggagctctcacatgtggaagctgcaaggtcttcttcaaaaga

SEQ ID NO: 78 - protein, 15 aa, Artificial Sequence
  Description of Artificial Sequence:variant 8 nuclear hormone receptor family zinc finger domain
YGALTCGSCKVFFKR

SEQ ID NO: 79 - DNA, 45 bp, Artificial Sequence
  Description of Artificial Sequence:variant 8 nuclear hormone receptor family zinc finger domain
tatggtgtccttacctgtgggagctgtaaggtcttctttaagagg

SEQ ID NO: 80 - protein, 15 aa, Artificial Sequence
  Description of Artificial Sequence:variant 8 nuclear hormone receptor family zinc finger domain
YGVLTCGSCKVFFKR

SEQ ID NO: 81 - DNA, 45 bp, Artificial Sequence
  Description of Artificial Sequence:variant 8 nuclear hormone receptor family zinc finger domain
tatggagtcttaacttgtggaagctgtaaagttttcttcaaaaga

SEQ ID NO: 82 - protein, 15 aa, Artificial Sequence
  Description of Artificial Sequence:variant 8 nuclear hormone receptor family zinc finger domain
YGVLTCGSCKVFFKR

SEQ ID NO: 83 - DNA, 45 bp, Artificial Sequence
  Description of Artificial Sequence:variant 9 nuclear hormone receptor family zinc finger domain
tacggcgtggcctcctgcgaggcttgcaaggccttcttcaagagg

SEQ ID NO: 84 - protein, 15 aa, Artificial Sequence
  Description of Artificial Sequence:variant 9 nuclear hormone receptor family zinc finger domain
YGVASCEACKAFFKR

SEQ ID NO: 85 - DNA, 45 bp, Artificial Sequence
  Description of Artificial Sequence:variant 9 nuclear hormone receptor family zinc finger domain
tatggtgtggcatcctgtgaggcctgcaaagccttcttcaagagg

SEQ ID NO: 86 - protein, 15 aa, Artificial Sequence
  Description of Artificial Sequence:variant 9 nuclear hormone receptor family zinc finger domain
YGVASCEACKAFFKR

SEQ ID NO: 87 - DNA, 45 bp, Artificial Sequence
  Description of Artificial Sequence:variant 9 nuclear hormone receptor family zinc finger domain
tatggagtctggtcctgtgagggctgcaaggccttcttcaagaga

SEQ ID NO: 88 - protein, 15 aa, Artificial Sequence
  Description of Artificial Sequence:variant 9 nuclear hormone receptor family zinc finger domain
YGVWSCEGCKAFFKR

SEQ ID NO: 89 - DNA, 45 bp, Artificial Sequence
  Description of Artificial Sequence:variant 9 nuclear hormone receptor family zinc finger domain
tatggagtctggtcgtgtgaaggatgtaaggccttttttaaaaga

SEQ ID NO: 90 - protein, 15 aa, Artificial Sequence
  Description of Artificial Sequence:variant 9 nuclear hormone receptor family zinc finger domain
YGVWSCEGCKAFFKR

SEQ ID NO: 91 - DNA, 48 bp, Artificial Sequence
  Description of Artificial Sequence:variant 1 tyrosine kinase family portion of catalytic domain
gttcccatcatccaccgcgaccttaagtccagcaacatattgatcctc

SEQ ID NO: 92 - protein, 16 aa, Artificial Sequence
  Description of Artificial Sequence:variant 1 tyrosine kinase family portion of catalytic domain
VPIIHRDLKSSNILIL

SEQ ID NO: 93 - DNA, 48 bp, Artificial Sequence
  Description of Artificial Sequence:variant 1 tyrosine kinase family portion of catalytic domain
gtgcccatcctgcaccgggacctcaagtccagcaacattttgctactt

SEQ ID NO: 94 - protein, 16 aa, Artificial Sequence
  Description of Artificial Sequence:variant 1 tyrosine kinase family portion of catalytic domain
VPILHRDLKSSNILLL

SEQ ID NO: 95 - DNA, 48 bp, Artificial Sequence; identical to SEQ ID NO: 93
  Description of Artificial Sequence:variant 1 tyrosine kinase family portion of catalytic domain

SEQ ID NO: 96 - protein, 16 aa, Artificial Sequence
  Description of Artificial Sequence:variant 1 tyrosine kinase family portion of catalytic domain
VPILHRDLKSSNILLL

SEQ ID NO: 97 - DNA, 48 bp, Artificial Sequence
  Description of Artificial Sequence:variant 2 tyrosine kinase family portion of catalytic domain
catggtatggtgcatagaaacctggctgcccgaaacgtgctactcaag

SEQ ID NO: 98 - protein, 16 aa, Artificial Sequence
  Description of Artificial Sequence:variant 2 tyrosine kinase family portion of catalytic domain
HGMVHRNLAARNVLLK

SEQ ID NO: 99 - DNA, 48 bp, Artificial Sequence
  Description of Artificial Sequence:variant 2 tyrosine kinase family portion of catalytic domain
aagaattgcatccaccgggacgtggcagcgcgtaacgtgctgttgacc

SEQ ID NO: 100 - protein, 16 aa, Artificial Sequence
  Description of Artificial Sequence:variant 2 tyrosine kinase family portion of catalytic domain
KNCIHRDVAARNVLLT

SEQ ID NO: 101 - DNA, 48 bp, Artificial Sequence
  Description of Artificial Sequence:variant 2 tyrosine kinase family portion of catalytic domain
atcaactgcgtgcacagggacattgctgtccggaacatcctggtggcc

SEQ ID NO: 102 - protein, 16 aa, Artificial Sequence
  Description of Artificial Sequence:variant 2 tyrosine kinase family portion of catalytic domain
INCVHRDIAVRNILVA

SEQ ID NO: 103 - DNA, 48 bp, Artificial Sequence
  Description of Artificial Sequence:variant 3 tyrosine kinase family portion of catalytic domain
atgaactacgtccaccgggaccttcgtgcagccaacatcctggtggga

SEQ ID NO: 104 - protein, 16 aa, Artificial Sequence
  Description of Artificial Sequence:variant 3 tyrosine kinase family portion of catalytic domain
MNYVHRDLRAANILVG

SEQ ID NO: 105 - DNA, 48 bp, Artificial Sequence
  Description of Artificial Sequence:variant 3 tyrosine kinase family portion of catalytic domain
atgaactatattcaccgagatcttcgggctgctaatattcttgtagga

SEQ ID NO: 106 - protein, 16 aa, Artificial Sequence
  Description of Artificial Sequence:variant 3 tyrosine kinase family portion of catalytic domain
MNYIHRDLRAANILVG

SEQ ID NO: 107 - DNA, 48 bp, Artificial Sequence
  Description of Artificial Sequence:variant 3 tyrosine kinase family portion of catalytic domain
atgaattatatccatagagatctgcgatcagcaaacattctagtgggg

SEQ ID NO: 108 - protein, 16 aa, Artificial Sequence
  Description of Artificial Sequence:variant 3 tyrosine kinase family portion of catalytic domain
MNYIHRDLRSANILVG

SEQ ID NO: 109 - DNA, 48 bp, Artificial Sequence
  Description of Artificial Sequence:variant 3 tyrosine kinase family portion of catalytic domain
atgaactacattcaccgcgacctgagggcagccaacatcctggttggg

SEQ ID NO: 110 - protein, 16 aa, Artificial Sequence
  Description of Artificial Sequence:variant 3 tyrosine kinase family portion of catalytic domain
MNYIHRDLRAANILVG

SEQ ID NO: 111 - DNA, 48 bp, Artificial Sequence
  Description of Artificial Sequence:variant 3 tyrosine kinase family portion of catalytic domain
aagaattccatccaccgcgacctgcgggcggccaacatcctggtgtct

SEQ ID NO: 112 - protein, 16 aa, Artificial Sequence
  Description of Artificial Sequence:variant 3 tyrosine kinase family portion of catalytic domain
MNSIHRDLRAANILVS

SEQ ID NO: 113 - DNA, 47 bp, Artificial Sequence
  Description of Artificial Sequence:variant 3 tyrosine kinase family portion of catalytic domain
aggaactacatccaccgagacctccgagctgccaacatcttggtctt

SEQ ID NO: 114 - protein, 16 aa, Artificial Sequence
  Description of Artificial Sequence:variant 3 tyrosine kinase family portion of catalytic domain
RNYIHRDLRAANILVS

SEQ ID NO: 115 - DNA, 48 bp, Artificial Sequence
  Description of Artificial Sequence:variant 3 tyrosine kinase family portion of catalytic domain
aagaactacattcaccgggacctgcgagcagctaatgttctggtctcc

SEQ ID NO: 116 - protein, 16 aa, Artificial Sequence
  Description of Artificial Sequence:variant 3 tyrosine kinase family portion of catalytic domain
KNYIHRDLRAANVLVS

SEQ ID NO: 117 - DNA, 48 bp, Artificial Sequence
  Description of Artificial Sequence:variant 3 tyrosine kinase family portion of catalytic domain
cggaattatattcatcgtgaccttcgggctgccaacattctggtgtct

SEQ ID NO: 118 - protein, 16 aa, Artificial Sequence
  Description of Artificial Sequence:variant 3 tyrosine kinase family portion of catalytic domain
RNYIHRDLRAANILVS

SEQ ID NO: 119 - DNA, 47 bp, Artificial Sequence
  Description of Artificial Sequence:variant 4 tyrosine kinase family portion of catalytic domain
ctgcattttgtgcaccgggacctggccacacgcaactgtctagtggg

SEQ ID NO: 120 - protein, 16 aa, Artificial Sequence
  Description of Artificial Sequence:variant 4 tyrosine kinase family portion of catalytic domain
LHFVHRDLATRNCLVG

SEQ ID NO: 121 - DNA, 48 bp, Artificial Sequence
  Description of Artificial Sequence:variant 4 tyrosine kinase family portion of catalytic domain
ctcaactttgtacatcgggacctggccacgcggaactgcctagttggg

SEQ ID NO: 122 - protein, 16 aa, Artificial Sequence
  Description of Artificial Sequence:variant 4 tyrosine kinase family portion of catalytic domain
LNFVHRDLATRNCLVG

SEQ ID NO: 123 - DNA, 48 bp, Artificial Sequence
  Description of Artificial Sequence:variant 4 tyrosine kinase family portion of catalytic domain
cttaattttgttcaccgagatctggccacacgaaactgtttagtgggt

SEQ ID NO: 124 - protein, 16 aa, Artificial Sequence
  Description of Artificial Sequence:variant 4 tyrosine kinase family portion of catalytic domain
LNFVHRDLATRNCLVG

SEQ ID NO: 125 - DNA, 48 bp, Artificial Sequence
  Description of Artificial Sequence:variant 4 tyrosine kinase family portion of catalytic domain
cgcgggctggtgcaccgagacctcgctacgcgcaacctactgctggcg

SEQ ID NO: 126 - protein, 16 aa, Artificial Sequence
  Description of Artificial Sequence:variant 4 tyrosine kinase family portion of catalytic domain
RGLVHRDLATRNLLLA

SEQ ID NO: 127 - DNA, 48 bp, Artificial Sequence
  Description of Artificial Sequence:variant 4 tyrosine kinase family portion of catalytic domain
aaaaggtatatccacagggatctggcaacgagaaatatattggtggag

SEQ ID NO: 128 - protein, 16 aa, Artificial Sequence
  Description of Artificial Sequence:variant 4 tyrosine kinase family portion of catalytic domain
KRYIHRDLATRNILVE

SEQ ID NO: 129 - DNA, 48 bp, Artificial Sequence
  Description of Artificial Sequence:variant 4 tyrosine kinase family portion of catalytic domain
cagcactttgtgcaccgagacctggccaccaggaactgcctggttgga

SEQ ID NO: 130 - protein, 16 aa, Artificial Sequence
  Description of Artificial Sequence:variant 4 tyrosine kinase family portion of catalytic domain
QHFVHRDLATRNCLVG

SEQ ID NO: 131 - DNA, 48 bp, Artificial Sequence
  Description of Artificial Sequence:variant 4 tyrosine kinase family portion of catalytic domain
cagcacttcgtgcaccgcgatttggccaccaggaactgcctggtcggg

SEQ ID NO: 132 - protein, 16 aa, Artificial Sequence
  Description of Artificial Sequence:variant 4 tyrosine kinase family portion of catalytic domain
QHFVHRDLATRNCLVG

SEQ ID NO: 133 - DNA, 48 bp, Artificial Sequence
  Description of Artificial Sequence:variant 4 tyrosine kinase family portion of catalytic domain
caccacgtggttcacaaggacctggccacccgcaatgtgctagtgtac

SEQ ID NO: 134 - protein, 16 aa, Artificial Sequence
  Description of Artificial Sequence:variant 4 tyrosine kinase family portion of catalytic domain
HHVVHKDLATRNVLVY

SEQ ID NO: 135 - DNA, 48 bp, Artificial Sequence
  Description of Artificial Sequence:variant 4 tyrosine kinase family portion of catalytic domain
cgtaagtttgttcaccgagatttagccaccaggaactgcctggtgggc

SEQ ID NO: 136 - protein, 16 aa, Artificial Sequence
  Description of Artificial Sequence:variant 4 tyrosine kinase family portion of catalytic domain
RKFVHRDLATRNCLVG

SEQ ID NO: 137 - DNA, 48 bp, Artificial Sequence
  Description of Artificial Sequence:variant 5 tyrosine kinase family portion of catalytic domain
aagaagcttgtgcaccgcgacctggccgcccgcaacatcctggtctca

SEQ ID NO: 138 - protein, 16 aa, Artificial Sequence
  Description of Artificial Sequence:variant 5 tyrosine kinase family portion of catalytic domain
KKLVHRDLAARNILVS

SEQ ID NO: 139 - DNA, 48 bp, Artificial Sequence
  Description of Artificial Sequence:variant 5 tyrosine kinase family portion of catalytic domain
aagaagcttgtgcaccgggacctagccgcccgcaacatcctggtctca

SEQ ID NO: 140 - protein, 16 aa, Artificial Sequence
  Description of Artificial Sequence:variant 5 tyrosine kinase family portion of catalytic domain
KKLVHRDLAARNILVS

SEQ ID NO: 141 - DNA, 48 bp, Artificial Sequence
  Description of Artificial Sequence:variant 5 tyrosine kinase family portion of catalytic domain
aacaatttcgtgcatcgagacctggctgcccgcaatgtgctggtgtct

SEQ ID NO: 142 - protein, 16 aa, Artificial Sequence
  Description of Artificial Sequence:variant 5 tyrosine kinase family portion of catalytic domain
NNFVHRDLAARNVLVS

SEQ ID NO: 143 - DNA, 48 bp, Artificial Sequence
  Description of Artificial Sequence:variant 5 tyrosine kinase family portion of catalytic domain
cacgactacatccaccgagacctagccgcgcgcaacgtgctgctggac

SEQ ID NO: 144 - protein, 16 aa, Artificial Sequence
  Description of Artificial Sequence:variant 5 tyrosine kinase family portion of catalytic domain
HDYIHRDLAARNVLLD

SEQ ID NO: 145 - DNA, 48 bp, Artificial Sequence
  Description of Artificial Sequence:variant 5 tyrosine kinase family portion of catalytic domain
cggcaatacgttcaccgggacttggcagcaagaaatgtccttgttgag

SEQ ID NO: 146 - protein, 16 aa, Artificial Sequence
  Description of Artificial Sequence:variant 5 tyrosine kinase family portion of catalytic domain
RQYVHRDLAARNVLVE

SEQ ID NO: 147 - DNA, 48 bp, Artificial Sequence
  Description of Artificial Sequence:variant 5 tyrosine kinase family portion of catalytic domain
cgtcgcttggtgcaccgcgacctggcagccaggaacgtactggtgaaa

SEQ ID NO: 148 - protein, 16 aa, Artificial Sequence
  Description of Artificial Sequence:variant 5 tyrosine kinase family portion of catalytic domain
RRLVHRDLAARNVLVK

SEQ ID NO: 149 - DNA, 48 bp, Artificial Sequence
  Description of Artificial Sequence:variant 5 tyrosine kinase family portion of catalytic domain
cggaacttcatccaccgagacctggctgctcggaattgcatgctggca

SEQ ID NO: 150 - protein, 16 aa, Artificial Sequence
  Description of Artificial Sequence:variant 5 tyrosine kinase family portion of catalytic domain
RNFIHRDLAARNCMLA

SEQ ID NO: 151 - DNA, 48 bp, Artificial Sequence
  Description of Artificial Sequence:variant 5 tyrosine kinase family portion of catalytic domain
aagaagtgcatacaccgagacctggcagccaggaatgtcctggtgaca

SEQ ID NO: 152 - protein, 16 aa, Artificial Sequence
  Description of Artificial Sequence:variant 5 tyrosine kinase family portion of catalytic domain
KKCIHRDLAARNVLVT

SEQ ID NO: 153 - DNA, 48 bp, Artificial Sequence
  Description of Artificial Sequence:variant 5 tyrosine kinase family portion of catalytic domain
caaaaatgtattcatcgagatttagcagccagaaatgttttggtaaca

SEQ ID NO: 154 - protein, 16 aa, Artificial Sequence
  Description of Artificial Sequence:variant 5 tyrosine kinase family portion of catalytic domain
QKCIHRDLAARNVLVT

SEQ ID NO: 155 - DNA, 48 bp, Artificial Sequence
  Description of Artificial Sequence:variant 5 tyrosine kinase family portion of catalytic domain
cagaagtgcatccacagggacctggctgcccgcaatgtgctggtgacc

SEQ ID NO: 156 - protein, 16 aa, Artificial Sequence
  Description of Artificial Sequence:variant 5 tyrosine kinase family portion of catalytic domain
QKCIHRDLAARNVLVT

SEQ ID NO: 157 - DNA, 48 bp, Artificial Sequence
  Description of Artificial Sequence:variant 5 tyrosine kinase family portion of catalytic domain
cagaagtgtattcacagagacttggctgccagaaacgtcctggtgacc

SEQ ID NO: 158 - protein, 16 aa, Artificial Sequence
  Description of Artificial Sequence:variant 5 tyrosine kinase family portion of catalytic domain
QKCIHRDLAARNVLVT

SEQ ID NO: 159 - DNA, 48 bp, Artificial Sequence
  Description of Artificial Sequence:variant 5 tyrosine kinase family portion of catalytic domain
cggaagtgtatccaccgggacctggctgcccgcaatgtgctggtgact

SEQ ID NO: 160 - protein, 16 aa, Artificial Sequence
  Description of Artificial Sequence:variant 5 tyrosine kinase family portion of catalytic domain
RKCIHRDLAARNVLVT

SEQ ID NO: 161 - DNA, 48 bp, Artificial Sequence
  Description of Artificial Sequence:variant 5 tyrosine kinase family portion of catalytic domain
atgaagctcgttcatcgggacttggcagccagaaacatcctggtagct

SEQ ID NO: 162 - protein, 16 aa, Artificial Sequence
  Description of Artificial Sequence:variant 5 tyrosine kinase family portion of catalytic domain
MKLVHRDLAARNILVA

SEQ ID NO: 163 - DNA, 48 bp, Artificial Sequence
  Description of Artificial Sequence:variant 5 tyrosine kinase family portion of catalytic domain
agaaagtgcattcatcgggacctggcagcgagaaacattcttttatct

SEQ ID NO: 164 - protein, 16 aa, Artificial Sequence
  Description of Artificial Sequence:variant 5 tyrosine kinase family portion of catalytic domain
RKCIHRDLAARNILLS

SEQ ID NO: 165 - DNA, 48 bp, Artificial Sequence
  Description of Artificial Sequence:variant 5 tyrosine kinase family portion of catalytic domain
cgaaagtgcatccacagagacctggctgctcggaacattctgctgtcg

SEQ ID NO: 166 - protein, 16 aa, Artificial Sequence
  Description of Artificial Sequence:variant 5 tyrosine kinase family portion of catalytic domain
RKCIHRDLAARNILLS

SEQ ID NO: 167 - DNA, 48 bp, Artificial Sequence
  Description of Artificial Sequence:variant 5 tyrosine kinase family portion of catalytic domain
cgaaagtgtatccacagggacctggcggcacgaaatatcctcttatcg

SEQ ID NO: 168 - protein, 16 aa, Artificial Sequence
  Description of Artificial Sequence:variant 5 tyrosine kinase family portion of catalytic domain
RKCIHRDLAARNILLS

SEQ ID NO: 169 - DNA, 48 bp, Artificial Sequence
  Description of Artificial Sequence:variant 5 tyrosine kinase family portion of catalytic domain
aagaactgcgtccacagagacctggcggctaggaacgtgctcatctgt

SEQ ID NO: 170 - protein, 16 aa, Artificial Sequence
  Description of Artificial Sequence:variant 5 tyrosine kinase family portion of catalytic domain
KNCVHRDLAARNVLIC

SEQ ID NO: 171 - DNA, 48 bp, Artificial Sequence
  Description of Artificial Sequence:variant 5 tyrosine kinase family portion of catalytic domain
aaaaattgtgtccaccgtgatctggctgctcgcaacgtcctcctggca

SEQ ID NO: 172 - protein, 16 aa, Artificial Sequence
  Description of Artificial Sequence:variant 5 tyrosine kinase family portion of catalytic domain
KNCVHRDLAARNVLLA

SEQ ID NO: 173 - DNA, 48 bp, Artificial Sequence
  Description of Artificial Sequence:variant 5 tyrosine kinase family portion of catalytic domain
aagaattgtattcacagagacttggcagccagaaatatcctccttact

SEQ ID NO: 174 - protein, 16 aa, Artificial Sequence
  Description of Artificial Sequence:variant 5 tyrosine kinase family portion of catalytic domain
KNCIHRDLAARNILLT

SEQ ID NO: 175 - DNA, 48 bp, Artificial Sequence
  Description of Artificial Sequence:variant 5 tyrosine kinase family portion of catalytic domain
aagtcgtgtgttcacagagacctggccgccaggaacgtgcttgtcacc

SEQ ID NO: 176 - protein, 16 aa, Artificial Sequence
  Description of Artificial Sequence:variant 5 tyrosine kinase family portion of catalytic domain
KSCVHRDLAARNVLVT

SEQ ID NO: 177 - DNA, 48 bp, Artificial Sequence
  Description of Artificial Sequence:variant 5 tyrosine kinase family portion of catalytic domain
aaacagtttattcacagggacctagctgccaggaacattttagttggt

SEQ ID NO: 178 - protein, 16 aa, Artificial Sequence
  Description of Artificial Sequence:variant 5 tyrosine kinase family portion of catalytic domain
KQFIHRDLAARNILVG

SEQ ID NO: 179 - DNA, 48 bp, Artificial Sequence
  Description of Artificial Sequence:variant 5 tyrosine kinase family portion of catalytic domain
aagcagttcatccacagggacctggctgcccggaatgtgctggtcgga

SEQ ID NO: 180 - protein, 16 aa, Artificial Sequence
  Description of Artificial Sequence:variant 5 tyrosine kinase family portion of catalytic domain
KQFIHRDLAARNVLVG

SEQ ID NO: 181 - DNA, 48 bp, Artificial Sequence; identical to SEQ ID NO: 179
  Description of Artificial Sequence:variant 5 tyrosine kinase family portion of catalytic domain

SEQ ID NO: 182 - protein, 16 aa, Artificial Sequence
  Description of Artificial Sequence:variant 5 tyrosine kinase family portion of catalytic domain
KQFIHRDLAARNVLVG

SEQ ID NO: 183 - DNA, 48 bp, Artificial Sequence
  Description of Artificial Sequence:variant 5 tyrosine kinase family portion of catalytic domain
atgaactatgtgcaccgtgacctggctgcccgcaacatcctcgtcaac

SEQ ID NO: 184 - protein, 16 aa, Artificial Sequence
  Description of Artificial Sequence:variant 5 tyrosine kinase family portion of catalytic domain
MNYVHRDLAARNILVN

SEQ ID NO: 185 - DNA, 48 bp, Artificial Sequence
  Description of Artificial Sequence:variant 5 tyrosine kinase family portion of catalytic domain
atgcatttcattcacagggatctggcagctagaaattgccttgtttcc

SEQ ID NO: 186 - protein, 16 aa, Artificial Sequence
  Description of Artificial Sequence:variant 5 tyrosine kinase family portion of catalytic domain
MHFIHRDLAARNCLVS

SEQ ID NO: 187 - DNA, 48 bp, Artificial Sequence
  Description of Artificial Sequence:variant 5 tyrosine kinase family portion of catalytic domain
aacaagtttgtgcaccgagatctagcagcccgcaactgcatggtgtcc

SEQ ID NO: 188 - protein, 16 aa, Artificial Sequence
  Description of Artificial Sequence:variant 5 tyrosine kinase family portion of catalytic domain
NKFVHRDLAARNCMVS

SEQ ID NO: 189 - DNA, 48 bp, Artificial Sequence
  Description of Artificial Sequence:variant 5 tyrosine kinase family portion of catalytic domain
aataagttcgtccacagagaccttgctgcccggaattgcatggtagcc

SEQ ID NO: 190 - protein, 16 aa, Artificial Sequence
  Description of Artificial Sequence:variant 5 tyrosine kinase family portion of catalytic domain
NKFVHRDLAARNCMVA

SEQ ID NO: 191 - DNA, 48 bp, Artificial Sequence
  Description of Artificial Sequence:variant 5 tyrosine kinase family portion of catalytic domain
aagaagtttgtgcatcgggacctggcagcgagaaactgcatggtcgcc

SEQ ID NO: 192 - protein, 16 aa, Artificial Sequence
  Description of Artificial Sequence:variant 5 tyrosine kinase family portion of catalytic domain
KKFVHRDLAARNCMVA

SEQ ID NO: 193 - DNA, 48 bp, Artificial Sequence
  Description of Artificial Sequence:variant 5 tyrosine kinase family portion of catalytic domain
aagagattcatacaccgggacctggcggccaggaactgcatgctgaat

SEQ ID NO: 194 - protein, 16 aa, Artificial Sequence
  Description of Artificial Sequence:variant 5 tyrosine kinase family portion of catalytic domain
KRFIHRDLAARNCMLN

SEQ ID NO: 195 - DNA, 48 bp, Artificial Sequence
  Description of Artificial Sequence:variant 5 tyrosine kinase family portion of catalytic domain
atgaactatgttcaccgtgacctggctgcccgcaacatcctcgtcaac

SEQ ID NO: 196 - protein, 16 aa, Artificial Sequence
  Description of Artificial Sequence:variant 5 tyrosine kinase family portion of catalytic domain
MNYVHRDLAARNILVN

SEQ ID NO: 197 - DNA, 48 bp, Artificial Sequence
  Description of Artificial Sequence:variant 5 tyrosine kinase family portion of catalytic domain
atgaactatgtgcaccgcgacctggctgctcgcaacatccttgtcaac

SEQ ID NO: 198 - protein, 16 aa, Artificial Sequence
  Description of Artificial Sequence:variant 5 tyrosine kinase family portion of catalytic domain
MNYVHRDLAARNILVN

SEQ ID NO: 199 - DNA, 48 bp, Artificial Sequence
  Description of Artificial Sequence:variant 5 tyrosine kinase family portion of catalytic domain
atgaattatgtgcatcgggacctggctgctaggaacattctggtcaac

SEQ ID NO: 200 - protein, 16 aa, Artificial Sequence
  Description of Artificial Sequence:variant 5 tyrosine kinase family portion of catalytic domain
MNYVHRDLAARNILVN

SEQ ID NO: 201 - DNA, 48 bp, Artificial Sequence
  Description of Artificial Sequence:variant 5 tyrosine kinase family portion of catalytic domain
atgggctatgtgcatagagatcttgctgccagaaacatcttaatcaac

SEQ ID NO: 202 - protein, 16 aa, Artificial Sequence
  Description of Artificial Sequence:variant 5 tyrosine kinase family portion of catalytic domain
MGYVHRDLAARNILIN

SEQ ID NO: 203 - DNA, 48 bp, Artificial Sequence
  Description of Artificial Sequence:variant 5 tyrosine kinase family portion of catalytic domain
cagaagtttgtgcacagggacctggctgcgcggaactgcatgctggac

SEQ ID NO: 204 - protein, 16 aa, Artificial Sequence
  Description of Artificial Sequence:variant 5 tyrosine kinase family portion of catalytic domain
QKFVHRDLAARNCMLD

SEQ ID NO: 205 - DNA, 48 bp, Artificial Sequence
  Description of Artificial Sequence:variant 5 tyrosine kinase family portion of catalytic domain
aaaaagtttgtccacagagacttggctgcaagaaactgtatgctggat

SEQ ID NO: 206 - protein, 16 aa, Artificial Sequence
  Description of Artificial Sequence:variant 5 tyrosine kinase family portion of catalytic domain
KKFVHRDLAARNCMLD

SEQ ID NO: 207 - DNA, 48 bp, Artificial Sequence
  Description of Artificial Sequence:variant 5 tyrosine kinase family portion of catalytic domain
atgggctatgttcaccgagacctcgctgctcggaacatcttgatcaac

SEQ ID NO: 208 - protein, 16 aa, Artificial Sequence
  Description of Artificial Sequence:variant 5 tyrosine kinase family portion of catalytic domain
MGYVHRDLAARNILIN

SEQ ID NO: 209 - DNA, 48 bp, Artificial Sequence
  Description of Artificial Sequence:variant 5 tyrosine kinase family portion of catalytic domain
aggaattttcttcatcgagatttagctgctcgaaactgcatgttgcga

SEQ ID NO: 210 - protein, 16 aa, Artificial Sequence
  Description of Artificial Sequence:variant 5 tyrosine kinase family portion of catalytic domain
RNFLHRDLAARNCMLR

SEQ ID NO: 211 - DNA, 48 bp, Artificial Sequence
  Description of Artificial Sequence:variant 5 tyrosine kinase family portion of catalytic domain
aaaaactgtatacacagggaccttgctgcaagaaactgcctggtaggt

SEQ ID NO: 212 - protein, 16 aa, Artificial Sequence
  Description of Artificial Sequence:variant 5 tyrosine kinase family portion of catalytic domain
KNCIHRDLAARNCLVG

SEQ ID NO: 213 - DNA, 48 bp, Artificial Sequence
  Description of Artificial Sequence:variant 5 tyrosine kinase family portion of catalytic domain
aagtgctgcatccaccgggacctggctgctcggaactgcctggtgaca

SEQ ID NO: 214 - protein, 16 aa, Artificial Sequence
  Description of Artificial Sequence:variant 5 tyrosine kinase family portion of catalytic domain
KCCIHRDLAARNCLVT

SEQ ID NO: 215 - DNA, 48 bp, Artificial Sequence
  Description of Artificial Sequence:variant 5 tyrosine kinase family portion of catalytic domain
atgagctatgtgcatcgtgatctggccgcacggaacatcctggtgaac

SEQ ID NO: 216 - protein, 16 aa, Artificial Sequence
  Description of Artificial Sequence:variant 5 tyrosine kinase family portion of catalytic domain
MSYVHRDLAARNILVN

SEQ ID NO: 217 - DNA, 48 bp, Artificial Sequence
  Description of Artificial Sequence:variant 5 tyrosine kinase family portion of catalytic domain
atgagctacgtccaccgagacctggctgctcgcaacatcctagtcaac

SEQ ID NO: 218 - protein, 16 aa, Artificial Sequence
  Description of Artificial Sequence:variant 5 tyrosine kinase family portion of catalytic domain
MSYVHRDLAARNILVN

SEQ ID NO: 219 - DNA, 48 bp, Artificial Sequence; identical to SEQ ID NO: 207
  Description of Artificial Sequence:variant 5 tyrosine kinase family portion of catalytic domain

SEQ ID NO: 220 - protein, 16 aa, Artificial Sequence
  Description of Artificial Sequence:variant 5 tyrosine kinase family portion of catalytic domain
MGYVHRDLAARNILIN

SEQ ID NO: 221 - DNA, 48 bp, Artificial Sequence
  Description of Artificial Sequence:variant 5 tyrosine kinase family portion of catalytic domain
atgggatatgttcacagggaccttgcagctcgcaatattcttgtcaac

SEQ ID NO: 222 - protein, 16 aa, Artificial Sequence
  Description of Artificial Sequence:variant 5 tyrosine kinase family portion of catalytic domain
MGYVHRDLAARNILVN

SEQ ID NO: 223 - DNA, 48 bp, Artificial Sequence; identical to SEQ ID NO: 147
  Description of Artificial Sequence:variant 5 tyrosine kinase family portion of catalytic domain

SEQ ID NO: 224 - protein, 16 aa, Artificial Sequence
  Description of Artificial Sequence:variant 5 tyrosine kinase family portion of catalytic domain
RRLVHRDLAARNVLVK

SEQ ID NO: 225 - DNA, 48 bp, Artificial Sequence
  Description of Artificial Sequence:variant 5 tyrosine kinase family portion of catalytic domain
gtgcggctcgtacacagggacttggccgctcggaacgtgctggtcaag

SEQ ID NO: 226 - protein, 16 aa, Artificial Sequence
  Description of Artificial Sequence:variant 5 tyrosine kinase family portion of catalytic domain
VRLVHRDLAARNVLVK

SEQ ID NO: 227 - DNA, 48 bp, Artificial Sequence
  Description of Artificial Sequence:variant 5 tyrosine kinase family portion of catalytic domain
agacgactcgttcatcgggatttggcagcccgtaatgtcttagtgaaa

SEQ ID NO: 228 - protein, 16 aa, Artificial Sequence
  Description of Artificial Sequence:variant 5 tyrosine kinase family portion of catalytic domain
RRLVHRDLAARNVLVK

SEQ ID NO: 229 - DNA, 48 bp, Artificial Sequence
  Description of Artificial Sequence:variant 5 tyrosine kinase family portion of catalytic domain
aaaaacttcatccacagagatcttgctgcccgaaactgcctggtaggg

SEQ ID NO: 230 - protein, 16 aa, Artificial Sequence
  Description of Artificial Sequence:variant 5 tyrosine kinase family portion of catalytic domain
KNFIHRDLAARNCLVG

SEQ ID NO: 231 - DNA, 48 bp, Artificial Sequence
  Description of Artificial Sequence:variant 5 tyrosine kinase family portion of catalytic domain
aagcgctttattcaccgtgacctggctgcccgcaatctgctgttggct

SEQ ID NO: 232 - protein, 16 aa, Artificial Sequence
  Description of Artificial Sequence:variant 5 tyrosine kinase family portion of catalytic domain
KRFIHRDLAARNLLLA

SEQ ID NO: 233 - DNA, 48 bp, Artificial Sequence
  Description of Artificial Sequence:variant 5 tyrosine kinase family portion of catalytic domain
aagaactttgtgcaccgtgacctggcggcccgcaacgtcctgctggtt

SEQ ID NO: 234 - protein, 16 aa, Artificial Sequence
  Description of Artificial Sequence:variant 5 tyrosine kinase family portion of catalytic domain
KNFVHRDLAARNVLLV

SEQ ID NO: 235 - DNA, 48 bp, Artificial Sequence; identical to SEQ ID NO: 233
  Description of Artificial Sequence:variant 5 tyrosine kinase family portion of catalytic domain

SEQ ID NO: 236 - protein, 16 aa, Artificial Sequence
  Description of Artificial Sequence:variant 5 tyrosine kinase family portion of catalytic domain
KNFVHRDLAARNVLLV

SEQ ID NO: 237 - DNA, 48 bp, Artificial Sequence
  Description of Artificial Sequence:variant 5 tyrosine kinase family portion of catalytic domain
agcaattttgtgcacagagatctggctgcaagaaatgtgttgctagtt

SEQ ID NO: 238 - protein, 16 aa, Artificial Sequence
  Description of Artificial Sequence:variant 5 tyrosine kinase family portion of catalytic domain
SNFVHRDLAARNVLLV

SEQ ID NO: 239 - DNA, 48 bp, Artificial Sequence
  Description of Artificial Sequence:variant 5 tyrosine kinase family portion of catalytic domain
cagaattacatccaccgggacctggccgccaggaacatcctcgtcggg

SEQ ID NO: 240 - protein, 16 aa, Artificial Sequence
  Description of Artificial Sequence:variant 5 tyrosine kinase family portion of catalytic domain
QNYIHRDLAARNILVG

SEQ ID NO: 241 - DNA, 48 bp, Artificial Sequence
  Description of Artificial Sequence:variant 5 tyrosine kinase family portion of catalytic domain
cagcgcgttgtgcaccgggacttggccgcccggaacgtgctcgtggac

SEQ ID NO: 242 - protein, 16 aa, Artificial Sequence
  Description of Artificial Sequence:variant 5 tyrosine kinase family portion of catalytic domain
QRVVHRDLAARNVLVD

SEQ ID NO: 243 - DNA, 48 bp, Artificial Sequence
  Description of Artificial Sequence:variant 5 tyrosine kinase family portion of catalytic domain
cggaactacattcacagagatctggctgccagaaatgtcctcgttggt

SEQ ID NO: 244 - protein, 16 aa, Artificial Sequence
  Description of Artificial Sequence:variant 5 tyrosine kinase family portion of catalytic domain
RNYIHRDLAARNVLVG

SEQ ID NO: 245 - DNA, 48 bp, Artificial Sequence
  Description of Artificial Sequence:variant 5 tyrosine kinase family portion of catalytic domain
aagaatttcatccatagagatcttgcagctcgtaactgcctagtggga

SEQ ID NO: 246 - protein, 16 aa, Artificial Sequence
  Description of Artificial Sequence:variant 5 tyrosine kinase family portion of catalytic domain
KNFIHRDLAARNCLVG

SEQ ID NO: 247 - DNA, 48 bp, Artificial Sequence
  Description of Artificial Sequence:variant 5 tyrosine kinase family portion of catalytic domain
aacagcttcatccacagagatctggctgccagaaattgtctagtaagt

SEQ ID NO: 248 - protein, 16 aa, Artificial Sequence
  Description of Artificial Sequence:variant 5 tyrosine kinase family portion of catalytic domain
NSFIHRDLAARNCLVS

SEQ ID NO: 249 - DNA, 48 bp, Artificial Sequence
  Description of Artificial Sequence:variant 5 tyrosine kinase family portion of catalytic domain
aatggctatattcatagggatttggcggcaaggaattgtttggtcagt

SEQ ID NO: 250 - protein, 16 aa, Artificial Sequence
  Description of Artificial Sequence:variant 5 tyrosine kinase family portion of catalytic domain
NGYIHRDLAARNCLVS

SEQ ID NO: 251 - DNA, 48 bp, Artificial Sequence
  Description of Artificial Sequence:variant 5 tyrosine kinase family portion of catalytic domain
gcatgtgtcatccacagagacttggctgccagaaattgtttggtggga

SEQ ID NO: 252 - protein, 16 aa, Artificial Sequence
  Description of Artificial Sequence:variant 5 tyrosine kinase family portion of catalytic domain
ACVIHRDLAARNCLVG

SEQ ID NO: 253 - DNA, 48 bp, Artificial Sequence
  Description of Artificial Sequence:variant 5 tyrosine kinase family portion of catalytic domain
caccaattcatacaccgggacttggctgctcgtaactgcttggtggac

SEQ ID NO: 254 - protein, 16 aa, Artificial Sequence
  Description of Artificial Sequence:variant 5 tyrosine kinase family portion of catalytic domain
HQFIHRDLAARNCLVD

SEQ ID NO: 255 - DNA, 48 bp, Artificial Sequence
  Description of Artificial Sequence:variant 5 tyrosine kinase family portion of catalytic domain
aagcagttccttcaccgagacctggcagctcgaaactgtttggtaaac

SEQ ID NO: 256 - protein, 16 aa, Artificial Sequence
  Description of Artificial Sequence:variant 5 tyrosine kinase family portion of catalytic domain
KQFLHRDLAARNCLVN

SEQ ID NO: 257 - DNA, 48 bp, Artificial Sequence
  Description of Artificial Sequence:variant 5 tyrosine kinase family portion of catalytic domain
cacaattatgtccaccgggacctggctgccagaaacatcttggtgaat

SEQ ID NO: 258 - protein, 16 aa, Artificial Sequence
  Description of Artificial Sequence:variant 5 tyrosine kinase family portion of catalytic domain
HNYVHRDLAARNILVN

SEQ ID NO: 259 - DNA, 48 bp, Artificial Sequence
  Description of Artificial Sequence:variant 6 tyrosine kinase family portion of catalytic domain
agggaagtcatccacaaagacctggctgccaggaactgtgtcattgat

SEQ ID NO: 260 - protein, 16 aa, Artificial Sequence
  Description of Artificial Sequence:variant 6 tyrosine kinase family portion of catalytic domain
REVIHKDLAARNCVID

SEQ ID NO: 261 - DNA, 48 bp, Artificial Sequence
  Description of Artificial Sequence:variant 6 tyrosine kinase family portion of catalytic domain
aaccgctttgtgcataaggacttggctgcgcgtaactgcctggtcagt

SEQ ID NO: 262 - protein, 16 aa, Artificial Sequence
  Description of Artificial Sequence:variant 6 tyrosine kinase family portion of catalytic domain
NRFVHKDLAARNCLVS

SEQ ID NO: 263 - DNA, 48 bp, Artificial Sequence
  Description of Artificial Sequence:variant 6 tyrosine kinase family portion of catalytic domain
cacttctttgtccacaaggaccttgcagctcgcaatattttaatcgga

SEQ ID NO: 264 - protein, 16 aa, Artificial Sequence
  Description of Artificial Sequence:variant 6 tyrosine kinase family portion of catalytic domain
HFFVHKDLAARNILIG

SEQ ID NO: 265 - DNA, 48 bp, Artificial Sequence
  Description of Artificial Sequence:variant 7 tyrosine kinase family portion of catalytic domain
aatcacttcatccacagggatattgccgcccggaactgcctgctgagc

SEQ ID NO: 266 - protein, 16 aa, Artificial Sequence
  Description of Artificial Sequence:variant 7 tyrosine kinase family portion of catalytic domain
NHFIHRDIAARNCLLS

SEQ ID NO: 267 - DNA, 48 bp, Artificial Sequence
  Description of Artificial Sequence:variant 7 tyrosine kinase family portion of catalytic domain
aaccacttcatccaccgagacattgctgccagaaactgcctcttgacc

SEQ ID NO: 268 - protein, 16 aa, Artificial Sequence
  Description of Artificial Sequence:variant 7 tyrosine kinase family portion of catalytic domain
NHFIHRDIAARNCLLT

SEQ ID NO: 269 - DNA, 54 bp, Artificial Sequence
  Description of Artificial Sequence:variant 1 semi-randomized oligonucleotide for tyrosine kinase family portion of catalytic domain
  modified_base (1)..(1): n = c modified by 5′ phosphate
ncaggacgacaaaaagachtgygarggstgyaargghctttttaggcttttcgg

SEQ ID NO: 270 - DNA, 54 bp, Artificial Sequence
  Description of Artificial Sequence:variant 2 semi-randomized oligonucleotide for tyrosine kinase family portion of catalytic domain
  modified_base (1)..(1): n = c modified by 5′ phosphate
  modified_base (37)..(37): n = g, a, c or t
ncaggacgacaaaaagwsytgygarggbtgcaarggnctttttaggcttttcgg

SEQ ID NO: 271 - DNA, 54 bp, Artificial Sequence
  Description of Artificial Sequence:variant 3 semi-randomized oligonucleotide for tyrosine kinase family portion of catalytic domain
  modified_base (1)..(1): n = c modified by 5′ phosphate
ncaggacgacaaaaagacstgcgagggctgcaaragyctttttaggcttttcgg

SEQ ID NO: 272 - DNA, 53 bp, Artificial Sequence
  Description of Artificial Sequence:variant 4 semi-randomized oligonucleotide for tyrosine kinase family portion of catalytic domain
  modified_base (1)..(1): n = c modified by 5′ phosphate
ncaggacgacaaaaagcctgcracggctgcwsmggyctttttaggcttttcgg

SEQ ID NO: 273 - DNA, 54 bp, Artificial Sequence
  Description of Artificial Sequence:variant 5 semi-randomized oligonucleotide for tyrosine kinase family portion of catalytic domain
  modified_base (1)..(1): n = c modified by 5′ phosphate
ncaggacgacaaaaagasctgtgayggstgcaagggyctttttaggcttttcgg

SEQ ID NO: 274 - DNA, 53 bp, Artificial Sequence
  Description of Artificial Sequence:variant 6 semi-randomized oligonucleotide for tyrosine kinase family portion of catalytic domain
  modified_base (1)..(1): n = c modified by 5′ phosphate
  modified_base (18)..(18): n = g, a, c or t
ncaggacgacaaaaagcntgygarggvtgyaagggyctttttaggcttttcgg

SEQ ID NO: 275 - DNA, 54 bp, Artificial Sequence
  Description of Artificial Sequence:variant 7 semi-randomized oligonucleotide for tyrosine kinase family portion of catalytic domain
  modified_base (1)..(1): n = c modified by 5′ phosphate
  modified_base (19)..(19): n = g, a, c or t
ncaggacgacaaaaagacntgtgarrgmtgcaaggghctttttaggcttttcgg

SEQ ID NO: 276 - DNA, 54 bp, Artificial Sequence
  Description of Artificial Sequence:variant 8 semi-randomized oligonucleotide for tyrosine kinase family portion of catalytic domain
  modified_base (1)..(1): n = c modified by 5′ phosphate
ncaggacgacaaaaagachtgtggvagctgyaargtyctttttaggcttttcgg

SEQ ID NO: 277 - DNA, 53 bp, Artificial Sequence
  Description of Artificial Sequence:variant 9 semi-randomized oligonucleotide for tyrosine kinase family portion of catalytic domain
  modified_base (1)..(1): n = c modified by 5′ phosphate
ncaggacgacaaaaagtcstgygargshtgyaargcctttttaggcttttcgg

SEQ ID NO: 278 - DNA, 20 bp, Artificial Sequence
  Description of Artificial Sequence:adapter oligonucleotide Univ-1(FseI)
ctttttgtcgtcctggccgg

SEQ ID NO: 279 - DNA, 21 bp, Artificial Sequence
  Description of Artificial Sequence:adapter oligonucleotide Univ-2(AscI)
  modified_base (1)..(1): n = c modified by 5′ phosphate
ngcgccgaaaagcctaaaaag

SEQ ID NO: 280 - DNA, 569 bp, Artificial Sequence
  Description of Artificial Sequence: humanU6/murine U6 opposing promoter cassette, human U6 and murine U6 promoters between TATA box and transcription start site modified to contain FseI and AscI restriction sites
ggatccaagcttaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcggccggcctcgaggcgcgccatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcc

SEQ ID NO: 281 - DNA, 21 bp, Artificial Sequence
  Description of Artificial Sequence:nuclear hormone receptor family zinc finger domain (ZnF_C4 domain) consensus sequence (21 nt)
  modified_base (1)..(21): n = g, a, c or t
dyntgyrrnrsntgywvvrbn

SEQ ID NO: 282 - protein, 7 aa, Artificial Sequence
  Description of Artificial Sequence:tyrosine kinase family portion of catalytic domain variant 1 signature motif
HRDLKSS

SEQ ID NO: 283 - protein, 7 aa, Artificial Sequence
  Description of Artificial Sequence:tyrosine kinase family portion of catalytic domain variant 2 signature motif
  MOD_RES (4): Xaa = Leu, Val or Ile
  MOD_RES (6): Xaa = Ala or Val
HRBXAXR

SEQ ID NO: 284 - protein, 7 aa, Artificial Sequence
  Description of Artificial Sequence:tyrosine kinase family portion of catalytic domain variant 3 signature motif
  MOD_RES (6): Xaa = Ala or Ser
HRDLRXA

SEQ ID NO: 285 - protein, 7 aa, Artificial Sequence
  Description of Artificial Sequence:tyrosine kinase family portion of catalytic domain variant 4 signature motif
  MOD_RES (2): Xaa = Arg or Lys
HXDLATR

SEQ ID NO: 286 - protein, 7 aa, Artificial Sequence
  Description of Artificial Sequence:tyrosine kinase family portion of catalytic domain variant 5 signature motif
HRDLAAR

SEQ ID NO: 287 - protein, 7 aa, Artificial Sequence
  Description of Artificial Sequence:tyrosine kinase family portion of catalytic domain variant 6 signature motif
HKDLAAR

SEQ ID NO: 288 - protein, 7 aa, Artificial Sequence
  Description of Artificial Sequence:tyrosine kinase family portion of catalytic domain variant 7 signature motif
HRDIAAR

SEQ ID NO: 289 - DNA, 19 bp, Artificial Sequence
  Description of Artificial Sequence:nuclear hormone receptor family zinc finger domain (ZnF_C4 domain) consensus sequence (19 nt)
  modified_base (1)..(19): n = g, a, c or t
dyntgyrrnrsntgywvvr

SEQ ID NO: 290 - DNA, 21 bp, Artificial Sequence
  Description of Artificial Sequence:tyrosine kinase family portion of catalytic domain variant 1 consensus sequence
caccgsgacctyaagtccagc

SEQ ID NO: 291 - DNA, 21 bp, Artificial Sequence
  Description of Artificial Sequence:tyrosine kinase family portion of catalytic domain variant 2 consensus sequence
caymgrracvtkgcwgyscgd

SEQ ID NO: 292 - DNA, 21 bp, Artificial Sequence
  Description of Artificial Sequence:tyrosine kinase family portion of catalytic domain variant 3 consensus sequence
  modified_base (9): n = g, a, c or t
caymgngayctbmgdkcdgch

SEQ ID NO: 293 - DNA, 21 bp, Artificial Sequence
  Description of Artificial Sequence:tyrosine kinase family portion of catalytic domain variant 4 consensus sequence
cacmrvgayytvgchacvmgv

SEQ ID NO: 294 - DNA, 21 bp, Artificial Sequence
  Description of Artificial Sequence:tyrosine kinase family portion of catalytic domain variant 5 consensus sequence
  modified_base (1)..(21): n = g, a, c or t
caymgngayytngcngcnmgn

SEQ ID NO: 295 - DNA, 21 bp, Artificial Sequence
  Description of Artificial Sequence:tyrosine kinase family portion of catalytic domain variant 6 consensus sequence
cayaargacytkgcwgcbmgb

SEQ ID NO: 296 - DNA, 21 bp, Artificial Sequence
  Description of Artificial Sequence:tyrosine kinase family portion of catalytic domain variant 7 consensus sequence
cacmgrgayattgcygccmgr

SEQ ID NO: 297 - protein, 7 aa, Artificial Sequence
  Description of Artificial Sequence:nuclear hormone receptor family zinc finger domain (ZnF_C4 domain) variant 1 signature motif
TCEGCKG

SEQ ID NO: 298 - DNA, 21 bp, Artificial Sequence
  Description of Artificial Sequence:nuclear hormone receptor family zinc finger domain (ZnF_C4 domain) variant 1 consensus sequence
achtgygarggstgyaarggh

SEQ ID NO: 299 - protein, 7 aa, Artificial Sequence
  Description of Artificial Sequence:nuclear hormone receptor family zinc finger domain (ZnF_C4 domain) variant 2 signature motif
SCEGCKG

SEQ ID NO: 300 - DNA, 21 bp, Artificial Sequence
  modified_base (21): n = g, a, c or t
wsytgygarggbtgcaarggn

SEQ ID NO: 301 - protein, 7 aa, Artificial Sequence
  Description of Artificial Sequence:nuclear hormone receptor family zinc finger domain (ZnF_C4 domain) variant 3 signature motif
TCEGCKS

SEQ ID NO: 302 - DNA, 21 bp, Artificial Sequence
  Description of Artificial Sequence:nuclear hormone receptor family zinc finger domain (ZnF_C4 domain) variant 3 consensus sequence
acstgcgagggctgcaaragy

SEQ ID NO: 303 - protein, 7 aa, Artificial Sequence
  Description of Artificial Sequence:nuclear hormone receptor family zinc finger domain (ZnF_C4 domain) variant 4 signature motif
ACBGCSG

SEQ ID NO: 304 - DNA, 21 bp, Artificial Sequence
  Description of Artificial Sequence:nuclear hormone receptor family zinc finger domain (ZnF_C4 domain) variant 4 consensus sequence
gcctgcracggctgcwsmggy

SEQ ID NO: 305 - protein, 7 aa, Artificial Sequence
  Description of Artificial Sequence:nuclear hormone receptor family zinc finger domain (ZnF_C4 domain) variant 5 signature motif
  MOD_RES (1): Xaa = Thr or Ser
XCDGCKG

SEQ ID NO: 306 - DNA, 21 bp, Artificial Sequence
  Description of Artificial Sequence:nuclear hormone receptor family zinc finger domain (ZnF_C4 domain) variant 5 consensus sequence
asctgtgayggstgcaagggy

SEQ ID NO: 307 - protein, 7 aa, Artificial Sequence
  Description of Artificial Sequence:nuclear hormone receptor family zinc finger domain (ZnF_C4 domain) variant 6 signature motif
ACEGCKG

SEQ ID NO: 308 - DNA, 21 bp, Artificial Sequence
  Description of Artificial Sequence:nuclear hormone receptor family zinc finger domain (ZnF_C4 domain) variant 6 consensus sequence
  modified_base (3): n = g, a, c or t
gcntgygarggvtgyaagggy

SEQ ID NO: 309 - protein, 7 aa, Artificial Sequence
  Description of Artificial Sequence:nuclear hormone receptor family zinc finger domain (ZnF_C4 domain) variant 7 signature motif
  MOD_RES (4): Xaa = Gly or Ser
TCEXCKG

SEQ ID NO: 310 - DNA, 21 bp, Artificial Sequence
  Description of Artificial Sequence:nuclear hormone receptor family zinc finger domain (ZnF_C4 domain) variant 7 consensus sequence
  modified_base (3): n = g, a, c or t
acntgtgarrgmtgcaagggh

SEQ ID NO: 311 - protein, 7 aa, Artificial Sequence
  Description of Artificial Sequence:nuclear hormone receptor family zinc finger domain (ZnF_C4 domain) variant 8 signature motif
TCGSCKV

SEQ ID NO: 312 - DNA, 21 bp, Artificial Sequence
  Description of Artificial Sequence:nuclear hormone receptor family zinc finger domain (ZnF_C4 domain) variant 8 consensus sequence
achtgtggvagctgyaargth

SEQ ID NO: 313 - protein, 7 aa, Artificial Sequence
  Description of Artificial Sequence:nuclear hormone receptor family zinc finger domain (ZnF_C4 domain) variant 9 signature motif
  MOD_RES (4): Xaa = Ala or Gly
SCEXCKA

SEQ ID NO: 314 - DNA, 21 bp, Artificial Sequence
  Description of Artificial Sequence:nuclear hormone receptor family zinc finger domain (ZnF_C4 domain) variant 9 consensus sequence
tcstgygargshtgyaargcc

(19) United States
(12) Patent Application Publication
(10) Pub. No.: US 2005/0026172 A1
(43) Pub. Date: Feb. 3, 2005

(54) siRNA libraries optimized for predetermined protein families

(75) Inventors: Henry Li, Carlsbad, CA (US); Jon E. Chatterton, San Diego, CA (US); Wufang Fan, San Diego, CA (US); Ning Ke, San Diego, CA (US); Flossie Wong-Staal, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Immusol, Inc., San Diego, CA (US)

(21) Appl. No.: 10/776,399

(22) Filed: Feb. 10, 2004

Related U.S. Application Data
(60) Provisional application No. 60/446,714, filed on Feb. 11, 2003.

(51) Int. Cl. C12Q 1/68; C07H 21/02

(52) U.S. Cl. 435/6; 536/23.1

(57) ABSTRACT

Libraries for generating small interfering RNA (siRNA) are provided where the members of the library are optimized to inhibit the expression of genes that encode a predetermined family of proteins. The members of the library target at least mRNA encoding all members of the family of proteins. Methods for generating siRNA libraries of the present invention are also provided.

### CROSS-REFERENCES TO RELATED APPLICATIONS

This application claims the benefit of U.S. Provisional Patent Application No. 60/446,714, filed on Feb. 11, 2003, the teachings of which are herein incorporated by reference.

### STATEMENT AS TO RIGHTS TO INVENTIONS MADE UNDER FEDERALLY SPONSORED RESEARCH OR DEVELOPMENT

Not Applicable

### BACKGROUND OF THE INVENTION

Small interfering RNAs (siRNA) are short double-stranded RNA fragments that elicit a process known as RNA interference (RNAi), a form of sequence-specific gene silencing. Zamore, Phillip et al., Cell 101:25-33 (2000); Elbashir, Sayda M., et al., Nature 411:494-497 (2001). siRNAs are assembled into a multicomponent complex known as the RNA-induced silencing complex (RISC). The siRNAs guide RISC to homologous mRNAs, targeting them for destruction. Hammond et al., Nature Genetics Reviews 2:110-119 (2000). RNAi has been observed in a variety of organisms including plants, insects and mammals. Since RNAi provides a means to specifically inhibit the expression of a gene by causing the rapid degradation of the mRNA of the gene, much research is now being conducted to ascertain if it is possible to use RNAi as a therapeutic tool, i.e. as a means to target and selectively silence specific genes known to be involved in various disease processes. RNAi is also being used as a research tool in the field of functional genomics, i.e. as a means for identifying and discovering hitherto unknown genes involved in disease processes, utilizing gene discovery techniques such as Inverse Genomics® which was developed by the Assignee hereof(see, e.g., WO 00/05415).

Various methods are known for the production of expression cassettes capable of expressing a library of siRNAs. In co-pending applications assigned to the Assignee hereof (U.S. Ser. Nos. 10/628,587 and 10/626,512), there are described methods for the expression of siRNAs in which all or most of the siRNA nucleotide sequence is fully randomized. For siRNAs having a length of 21 nucleotides, the fully random siRNA library contains (4²¹)/2 or 2.2×10¹² unique members. A library of such size (“complexity”) is very useful for purposes of gene discovery utilizing the techniques of Inverse Genomics®, but there are certain practical drawbacks inherent in the use of a library of such complexity. Under certain circumstances, using a library of such complexity may be unnecessary and even counter-productive. For example, if it is desired to study the effect of RNAi on a small number of genes known to encode a family of proteins, it would be preferable to express a more limited (less complex) library that comprises only the siRNA that silences these genes, rather than a totally randomized library of full complexity. Heretofore, it was impossible to do so, and the only alternative was to synthesize individually each and every siRNA of interest.

The inventors hereof have now discovered a method for expressing a library of siRNAs wherein the library is optimized to include at least all siRNAs which functionally silence specific genes of interest, e.g. genes which encode a predetermined family of proteins. This novel method is highly advantageous over other methods currently known or practiced in the art. It allows for the molecular cloning of the entire targeted library of siRNAs of interest in a single step, thereby eliminating the relatively high cost and time-consumption involved in the synthesis of individual siRNAs. It also allows for the delivery of the siRNAs in a pooled fashion, making it possible to do combinatorial screening without need for more expensive robot-based high-throughput screening methods. In addition, it provides a high degree of flexibility in the design and expression of the library of interest, making it possible to modify easily the complexity of the library (i.e., increase or decrease its size) depending upon the goals of the research and the information that is available with respect to the genes or protein family of interest. Finally, since the siRNA libraries of the present invention are expressed by means of partially randomized gene sequences, they comprise not only siRNAs having the ability to silence genes encoding all the known members of a protein family of interest but additional genes as well, thereby expanding the possibilities (via techniques such as Inverse Genomics®) for discovery of novel genes heretofore not known to express proteins belonging to the family of interest.

### BRIEF SUMMARY OF THE INVENTION

The present invention provides an siRNA expression library for selective post-transcriptional silencing of genes encoding a family of proteins, wherein members of the library encode siRNA molecules that are of between 15 to 30 nucleotides in length and target at least all mRNAs encoding all known members of the family of proteins. The library may comprise between 50 and one million unique members. In a preferred embodiment, the siRNA molecules are between 18 to 24 nucleotides in length. In yet another preferred embodiment, the family of proteins is any that is known to be involved in disease processes, such as G protein coupled receptors, ion channels, receptor tyrosine kinases, non-receptor tyrosine kinases, nuclear hormone receptors, GTPases, ATPases, serine/threonine kinases, proteases, matrix metalloproteinases (MMPs), GTPase-activating proteins (GAPs), E3 ubiquitin ligases, or others.

The present invention also provides a method for generating an siRNA expression library for selective post-transcriptional silencing of genes encoding a family of proteins, the method comprising identifying a consensus sequence for the family of proteins and generating an siRNA expression library whose members encode siRNA molecules that target at least all mRNAs encoding all known members of the family of proteins. The consensus sequence may comprise between 15 to 30 nucleotides, and preferably, between 18 to 24 nucleotides. In one embodiment, the consensus sequence is determined after identifying at least one signature motif for the family of proteins. In another embodiment, two or more variants of a signature motif for the family of proteins are identified, and a consensus sequence is determined for each of the variants.

### BRIEF DESCRIPTION OF THE DRAWING

FIG. 1 depicts an exemplary DNA expression cassette for expressing the siRNA from opposing pol III promoters (U6 promoters shown) in accordance with the present invention.

### DEFINITIONS

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” or “cellular gene” refers to a nucleic acid fragment that encodes a specific transcription product; it includes regions preceding (5′ non-coding) and following (3′ non-coding) the coding region that control transcriptional expression as well as intervening sequences (introns) between individual coding segments (exons).

The term “dsRNA,” or double-stranded RNA, refers to an RNA molecule comprising two hybridized complementary RNA strands in a double-stranded conformation through base pairing interactions. The term “siRNA” refers to a dsRNA that is preferably between 15 and 30, and more preferably between 18 and 24 base pairs long, each strand of which can have a short 3′ overhang. Functionally, the characteristic distinguishing an siRNA over other forms of dsRNA is that an siRNA is capable of specifically inhibiting expression of a gene by a process termed “RNA interference” (RNAi), and, due to their small size, do not induce in mammalian cells the interferon and PKR pathways that can lead to non-specific inhibition of gene expression.

A “library” as used herein refers to a collection of nucleic acid sequences that possesses a common characteristic. For example, a library of nucleic acids can be representative of all possible configurations of a nucleic acid sequence over a defined length. Alternatively, a nucleic acid library may be a collection of sequences that represents a particular subset of the possible sequence configurations of a nucleic acid of a defined length. A library may also represent all or part of the genetic information of a particular organism. A nucleic acid “library” is typically, but not necessarily, cloned into a vector.

An “siRNA expression library” of the invention is a nucleic acid library that is capable of generating a collection of siRNA molecules by a transcription process.

“Polypeptide,” “peptide,” and “protein” are used interchangeably herein to refer to a polymer of amino acid residues. All three terms apply to amino acid polymers in which one or more amino acid residues are an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

A “family of proteins” as used herein refers to two or more proteins that carry out similar or related biochemical functions. The members of a family of proteins demonstrate a substantial level of amino acid sequence homology in at least one conserved domain which typically relates to the functional characteristics of the family. A “family of genes” consists of the genes that encode a family of proteins.

A “signature motif” as used herein refers to an amino acid sequence characteristic for the members of a family of proteins and is typically found within a highly conserved domain critical for the biological functions of the family of proteins. The length of a signature motif is preferably 5-10, and more preferably 6-8, amino acids. Among the amino acids of a signature motif, typically about 50%, preferably about 60% or more, are constant within all members of the family and the balance are variable. For certain families of proteins, the practice of the present invention may involve the identification of two or more variants of a signature motif, each variant representing the amino acid sequences of a sub-set of the proteins comprising the family.

The term “consensus sequence” as used herein defines the set of nucleic acid sequences that encodes the amino acid sequences of at least all members of a family of proteins sharing the same signature motif. Typically, there are multiple nucleotide sequences that encode the amino acid sequences of a signature motif, due to both the variability in amino acid sequence within the signature motif itself, and codon degeneracy. A consensus sequence is represented by a formula comprising both constant and variable bases. Among the variable bases, some may be “fully random” (or “random”), i.e., they may be any of the four possible bases. Others may be “partially random”, i.e., they may comprise only two or only three predetermined bases of the four possible bases. The length of a consensus sequence may vary depending on the length of the signature motif. Typically, the length is between 15-30 nucleotides; more frequently, between 18-24 nucleotides.

Amino acids may be referred to herein by either the commonly known three-letter symbols or by the one-letter symbols recommended by the IUPAC-IUB Biochemical Nomenclature Commission. Nucleotides, likewise, may be referred to by their commonly accepted single-letter codes.

The term “gene expression” as used herein refers to all processes involved in producing a biologically active agent, which may be a nucleic acid (e.g., an mRNA) or protein (e.g., an enzyme) in nature, from a nucleic acid encoding the biologically active agent. Gene expression includes all post-transcriptional (e.g., RNA splicing) and/or post-translational processing (e.g., post-translational modification such as glycosylation) required to produce the mature agent. Gene expression may be “silenced,” “inhibited” or “suppressed” by any means that interrupts the process leading to the production of the biologically active agent, including interruptions at transcriptional, post-transcriptional, translational, and post-translational levels. For the purpose of the present invention, “post-transcriptional gene silencing” refers to the effect of the siRNA produced in accordance with the invention in suppressing the expression of genes encoding proteins belonging to a family of proteins of interest.

The term “sense siRNA strand” refers to the siRNA strand that matches the target mRNA sequence. The term “antisense siRNA strand” refers to the siRNA strand that is complementary to the target mRNA sequence.

### DETAILED DESCRIPTION OF THE INVENTION

I. Introduction

The present invention provides a novel method for designing and expressing a library of siRNAs wherein the library is optimized to include at least all siRNAs sufficient to functionally silence the genes which encode all members of a predetermined family of proteins. The invention provides for the molecular cloning of the entire library of siRNAs of interest in a single step, and eliminates the high cost involved in the synthesis of individual siRNAs. The method also affords a high degree of flexibility in the design and expression of an siRNA library, allowing the researcher to easily modify the complexity of the library (i.e. increase or decrease its size), depending upon the goals of the research and the information that is available with respect to the genes or protein family of interest. The invention has particular application in genomics research, and may be effectively used in connection with the identification and validation of genes coding for proteins which are known or suspected to be involved in disease processes, including G protein coupled receptors, ion channels, receptor tyrosine kinases, non-receptor tyrosine kinases, nuclear hormone receptors, GTPases, ATPases, serine/threonine kinases, proteases, matrix metalloproteinases (MMPs), GTPase-activating proteins (GAPs), and E3 ubiquitin ligases. Although from a theoretical standpoint a library of the present invention need not be limited in size, practical considerations dictate designing a library with more limited complexity. Typically, a library designed and constructed in accordance with the invention will comprise between 20,000 and 100,000 members, although libraries having as few as 50 members or as many as one million members are also included within the scope of the invention.

II. Identification of a Signature Motif

The construction of an siRNA expression library in accordance with the present invention requires as a first step identifying at least one “signature motif ” for the family of proteins of interest. Each signature motif is an amino acid sequence characteristic for the members of the family of proteins and is usually found within a highly conserved domain critical for the biological functions of the members of the family. The highly conserved domain and signature motif may be identified by various means known in the art including alignment of amino acid and nucleotide sequences and analysis of sequence homology within the family. A. D. Baxevanis et al., Bioinformatics—A Practical Guide to the Analysis of Genes and Proteins. 2^{nd} ed. (1998). Various tools are available to assist in the identification of a signature motif, including software such as CLUSTALW (Higgens et al. 1996), which may be used with various default parameters, or modified as needed. A signature motif is typically 5-10 and more preferably 6-8 amino acids in length. Among the amino acids comprising a signature sequence, preferably about 50%, more preferably 60% or more, are constant within the members of the family of proteins and the balance are variable.

A representative signature motif for the family of nuclear hormone receptors is shown in Example 1. This is a signature motif located within the Zinc Finger_C4 domain of the 45 known members of this family of proteins and comprises the amino acid sequence: (T/S/A)-C-(D/E/G/N)-(G/S/A)-(C)-(K/S)-(A/G/S/V), where the second and fifth amino acids of the sequence, C (cysteine), are constant within all members of the family, and the balance are variable. It will be appreciated that the degree of variability of the remaining amino acids is not equal throughout this signature motif. Thus, the first and fourth positions may be filled by any of three amino acids, the third and seventh positions may be filled by any of four amino acids, and the sixth position may be filled by either of two amino acids.

For certain families of proteins, e.g., those with a very large number of members, or those for whom it may not be possible to identify a single signature motif across all members or for whom designing an siRNA expression library based upon a single signature motif would result in a library that would be functionally too complex, the practice of the present invention may involve the identification of two or more variants of a signature motif, with each variant representing the amino acid sequences characteristic of only a subset of the proteins comprising the family of proteins. A representative example is the family of tyrosine kinases which currently has 89 known members. As shown in Example 2, at least seven variants of a signature motif for this family may be identified, each variant representing a sub-set of the family as a whole, the sub-sets comprising as few as two members and as many as 61 members.

III. Determining a Consensus Sequence

Once a signature motif has been identified, as described above, the signature motif is then “reverse translated” into a “consensus sequence” representing the set of nucleic acid sequences that encodes the amino acid sequences of at least all the known proteins sharing the signature motif. The “reverse translation” process may be performed by deducing all possible codons for each amino acid in the signature motif from the genetic code or by extracting the specific coding sequence corresponding to the signature motif for each member of the family from an appropriate sequence database (e.g., Genbank). The length of a consensus sequence may vary depending on the length of the signature motif. Typically, the length is between 15-30 nucleotides; more preferably, between 18-24 nucleotides.

A consensus sequence may be represented by a formula, comprising both fixed and variable bases. Thus, the consensus sequence for the signature motif for the family of nuclear hormone receptors mentioned above and shown in Example 1 is:

[(A/T/G) (C/T) (A/G/T/C)] [TG(T/C)] [(A/G) (A/G) (A/C/G/T)]
[(A/G) (C/G) (A/C/G/T)] [TG(T/C)] [(A/T) (A/C/G) (A/C/G)]
[(A/G) (C/G/T) (A/C/G/T)]

As can be seen, among the variable bases, some may be fully random, i.e., they may be any of the four possible bases, A, C, G or T. Others may be partially random, i.e., they may comprise only two or only three predetermined bases of the four possible bases. Generally, in determining a consensus sequence, all possible codon variations for a given amino acid will be taken into account; however, for various reasons, including the need to limit the complexity (i.e. size) of the siRNA library, the consensus sequence may be restricted to include only the specific codons known to code for the amino acids comprising the known members of the protein family.

Once a consensus sequence has been determined for a family of proteins, as described above, DNA oligonucleotides may be chemically synthesized in a single batch for all nucleic acid sequences defined by the consensus sequence, and these may be utilized as siRNA coding sequences for incorporation into expression cassettes capable of expressing an siRNA library in accordance with the invention. It will be appreciated that the siRNA library expressed in this manner will be capable of silencing the genes encoding at least all known proteins within the predetermined family of proteins, although the library will also be capable of silencing additional genes which have not yet been identified or that do not exist in nature. Thus, in the above example, the signature motif was determined based upon the amino acid sequences of 45 known members of the family of nuclear hormone receptors. However, the siRNA library that may be expressed based upon the consensus sequence corresponding to this signature motif comprises a significantly larger number of members, due to the partial randomness of the nucleotide coding sequence. In the above example, since there are nine positions that may be filled by any of two bases, four positions that may be filled by any of three bases and four positions that may be filled by any of four bases, the total number of permutations represented by the consensus sequence is 2⁹×3⁴×4⁴, or 10,616,832. Thus, the siRNA library that will be expressed will have a complexity of 10,616,832 members, and will be capable of silencing not only the genes encoding the known members of the family of nuclear hormone receptors but also the genes encoding as yet unknown members of the family, as well as many other genes matching the consensus sequence, including genes that code for proteins in the other two reading frames and genes that are complementary to the consensus sequence.

IV. Expression Cassettes

Expression cassettes for expressing siRNA libraries in accordance with the invention may be constructed by any method known in the art, in particular, methods that allow for transcription of both strands of the double-stranded siRNA even when the coding sequence comprises partially randomized nucleotides, as is the case with the sequences defined by a consensus sequence in accordance with the present invention.

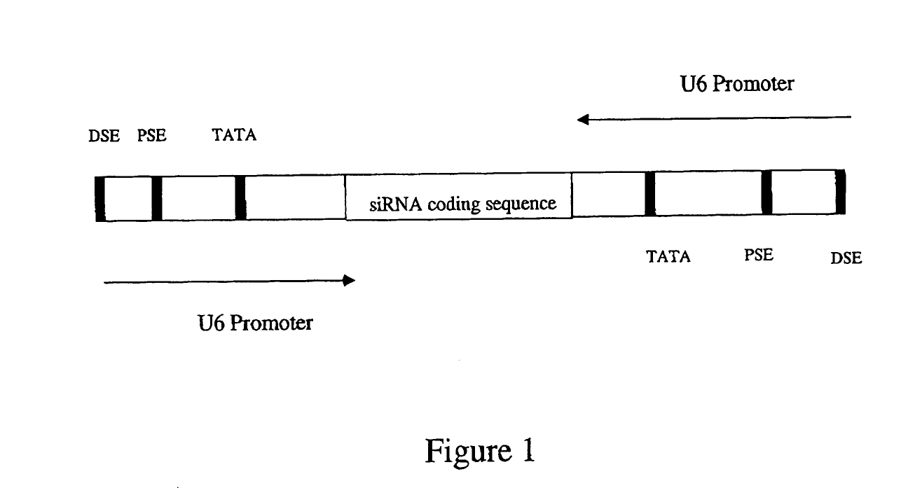

A particularly preferred method involves the use of a dual promoter system that allows for ligating the nucleic acid sequence encoding the siRNA between two suitable promoters oriented in opposite orientation. “Opposite orientation” refers to a positioning of the two promoters (see FIG. 1) such that one promoter will be operably linked to the “sense” strand of the nucleic acid and the other promoter operably linked to the “anti-sense” strand. When properly positioned, the promoters preferably initiate transcription at the first base encoding the siRNA of interest. Transcription terminates at a specific termination sequence which, when using the preferred pol III type III promoters described below, comprise at least four thymidyl residues located at the end of the siRNA coding sequence, preferably located in the 3′ end of the opposite promoter. In addition to a termination sequence, the expression cassette construct can optionally contain a restriction site to ease recovery of the sequence encoding the siRNA. This restriction site is preferably located 5′ to the four thymidyl residues and 3′ to the TATA box and created by substitution of existing bases of the promoter sequence, preferably using site-directed mutagenesis techniques as is known in the art. Anywhere from 0 to 20 bases can be modified in the region 5′ to the four thymidyl residues and 3′ to the TATA box, to create restriction sequences, operator sequences or other genetic or cloning elements. The nucleic acid encoding the antisense siRNA strand is synthesized, preferably enzymatically, after the nucleic acid encoding the sense siRNA strand is ligated between the oppositely orientated promoters. Alternatively, the nucleic acid encoding the antisense siRNA strand can be ligated between the oppositely oriented promoters and the nucleic acid encoding the sense siRNA strand can be subsequently synthesized enzymatically. Enzymatic methods for DNA oligonucleotide synthesis frequently employ Klenow, T7, T4, Taq or E. coli DNA polymerase as described in Sambrook and Russel, Molecular Cloning. A Laboratory Manual, 3^{rd} ed. (2001). Methods for construction of dual promoter siRNA expression cassettes are described in U.S. patent application Ser. No. 10/626,512, the teachings of which are incorporated herein by reference.

Alternatively, the expression cassettes may be constructed such that they express hairpin siRNAs (shRNAs) from a single promoter [e.g., Paddison, P. J. et al. Genes and Development, 16: 948-958 (2002); Brummelkamp, T. R. et al. Science, 296: 550-553 (2002)]. Methods for the construction of the hairpin siRNA expression cassettes from a partially randomized oligonucleotide are described in U.S. patent application Ser. No. 10/628,587, the teachings of which are incorporated herein by reference.

In another embodiment, the siRNA expression cassettes are constructed using the polymerase chain reaction (PCR). Those skilled in the art will recognize that functional pol III promoters can be operably linked to each end of an siRNA coding region by PCR [e.g., see Methods in Molecular Biology, Vol. 15: PCR Protocols: Current Methods and Applications. White, B. A., ed. Humana Press, Inc., Totowa, N.J. (1993)]. This approach requires the addition of oligonucleotide extensions to each end of the semi-randomized oligonucleotide to serve as priming sites. The sequence of the oligonucleotide extensions is dependent on the choice of pol III promoters.

The particular promoters chosen for use in the expression cassettes of the present invention will depend upon which organism or cell type is to be targeted by the siRNA encoded in the expression cassette. For example, if plant cells are to be the target, then plant promoters should be used. The promoters can be constitutive, inducible, or cell dependent, depending on the application and result desired. The promoters do not have to be the same, although they can be. They can be of different types, isolated from different genes, be differentially regulated or differ by as little as one base.

Preferably the promoters will not require any intragenic promoter elements, so as allow for the greatest degree of flexibility when designing the coding region of the cassette. The promoters will- also preferably not have a requirement for a particular nucleotide at the transcription start-point, although some specificity is tolerable, including a specific requirement for a G or A at the first position by some polymerases. Particularly preferred promoters meeting the above criteria are RNA polymerase III (pol III) promoters of type III, such as the human U6 small nuclear RNA gene promoter and the promoter for human H1 RNA. Such promoters can produce transcripts constitutively without cell type specific expression, although operator sequences can be engineered rendering the promoter inducible. The use of U6 gene transcription signals to produce short RNA molecules in vivo is described by Miyagishi and Taira, Nature Biotechnology, 20:497-500 (2002); Lee, Nan Sook, et al., Nature Biotechnology, 20:500-505 (2002); Noonberg et al., Nucleic Acids Res., 22:2830-2836 (1995), and the use of H1 RNA promoters is described by Baer et al., Nucleic Acids Res., 18:97-103 (1990) and Hannon et al., J. Biol. Chem., 266:22796-22799 (1991). The preferred promoters mentioned above, such as the U6 promoter and the human HI promoter contain all of the cis-acting promoter elements upstream of the transcription start site. These upstream sequence elements include a TATA box (Mattaj et al., Cell, 55:435-442 (1988)), a proximal sequence element (PSE), and in some circumstances a distal sequence element (DSE, Gupta and Reddy, Nucleic Acids Res., 19:2073-2075 (1991)), as shown in FIG. 1. Alternatively, tRNA promoters [Kawasaki and Taira, Nucl. Acids Res, 31: 700-707 (2003)] and pol II promoters [Xia, H. et al., Nat. Biotechnol., 20: 1006-1010 (2002)] may be used.

V. General Recombinant Methods for Constructing siRNA Libraries

The construction of expression cassettes suitable for practicing the present invention utilizes methods known to those skilled in the art of molecular biology. In general, the expression cassettes may be ligated into a DNA transfer vector, such as a plasmid, bacteriophage DNA, or lentiviral, adenoviral, alphaviral, or other viral vector. Prokaryotic or eukaryotic host cells may then be transfected or transduced with an appropriate transfer vector containing genetic material corresponding to an expression cassette in accordance with the present invention, such that the siRNA is transcribed in the host cells. The siRNA expression cassettes can also be delivered directly to the host cells by transfection without prior ligation into a DNA transfer vector [e.g., see Castanotto, D. et al., RNA 8: 1454-1460 (2002)].

In preparing the expression cassettes, the DNA sequences may be inserted or substituted into a bacterial plasmid. Any convenient plasmid may be employed, which will be characterized by having a bacterial replication system, a marker that allows for selection in the bacterium, and generally one or more unique, conveniently located restriction sites. These plasmids, referred to as vectors, may include such vectors as pACYC 184, pACYC 177, pBR322, pUC9, and their derivatives. A particular plasmid is often chosen based on the nature of the markers, the availability of convenient restriction sites, copy number, and the like. Subsequently, the DNA sequence encoding an siRNA, may be inserted into the vector at an appropriate restriction site, and the resulting plasmid is used to transform the E. coli host. After the transformed E. coli is cultured in an appropriate nutrient medium, the bacteria are harvested and lysed, and the plasmid recovered.

Basic texts disclosing the general methods for use in connection with this invention include Sambrook and Russell, Molecular Cloning: A Laboratory Manual 3d ed. (2001); Gelvin et al., eds. Plant Molecular Biology Manual (1990); Kriegler, Gene Transfer and Expression: A Laboratory Manual (1990); and Ausubel et al., Current Protocols in Molecular Biology (1994).

Chemical synthesis of linear oligonucleotides is well known in the art and can be made by any of several different synthetic procedures including the phosphoramidite, phosphite triester, H-phosphonate and phosphotriester methods, typically by automated synthesis methods. Beaucage and Caruthers, Tetrahedron Letts., 22:1859-1862 (1981); Needham-VanDevanter et al., Nucleic Acids Res., 12:6159-6168 (1984). Moreover, oligonucleotides can also be custom-made and ordered from a variety of commercial sources known to persons of skill in the art. It will be appreciated that in preparing the oligonucleotides in accordance with the invention, appropriate instructions are provided to the synthesizer with respect to the randomization of the nucleotides within the consensus sequence that are not fixed, such that each “wobble” position is randomly filled with one of the two or one of the three or one of the four nucleotides allowed for that position as stipulated by the consensus sequence.

The sequence of the isolated and synthetic oligonucleotides utilized in the practice of the present invention can be verified after cloning using, e.g., the chain termination method for sequencing double-stranded templates of Wallace et al., Gene, 16:21 -26 (1981).

VI. Reducing Library Complexity

As already indicated, the present invention provides a significant amount of flexibility with respect to the complexity (number of members) of the siRNA libraries produced in accordance with the invention. This flexibility is a result of the ability to modify a number of parameters involved in the design and construction of such libraries. Included among these parameters are the length of the signature motif and the number of amino acid positions within the signature motif that are constant for all members. Thus, a shorter signature motif (e.g. six amino acids rather than seven) or one that has a larger number of amino acids that are constant (e.g. five rather than three or four) will generally “reverse translate” into a consensus sequence having a larger percentage of bases that are constant, and as a consequence, a library generated on the basis of such a consensus sequence will have fewer members. Similarly, the complexity of a library may also be reduced by truncating the consensus sequence (e.g., by eliminating one or more nucleotide positions at either the 3′ end or 5′ end of the sequence, as illustrated in Example 1 below), or, as already indicated, by limiting the randomness of the nucleotides comprising a consensus sequence, by utilizing only those codons that encode for amino acid sequences of known members of the family of protiens of interest, rather than all possible codons based upon the degeneracy of the genetic code.

An additional and effective way to reduce the complexity of a library is to divide the members of a protein family of interest into two or more sub-sets, each sub-set comprising members having a variant of the signature sequence, each such variant comprising a relatively high number of amino acids that are constant for all members of the sub-set effect of such division can be seen clearly with reference to Example 2 and Table 1 below, which shows the division into seven sub-sets of the 89 known members of the family of tyrosine kinases. Each of sub-sets 1 and 4-7 have a different variant of the signature motif, but all five comprise seven amino acids that are constant for all members of the respective sub-set. Sub-set 3 has a variant signature sequence in which only one of the seven amino acids is not constant for all members of the sub-set; and only sub-set 2 has a variant signature motif in which three of the amino acids are not constant for all members.

TABLE 1

| | | | | | | | | No. of Known | |
|---|---|---|---|---|---|---|---|---|---|
| Variant | Signature Motif | | | | | | | Members | Complexity |
| 1 | H | R | D | L | K | S | S | 3 | 4 |
| 2 | H | R | N/D | L/V/I | A | A/V | R | 3 | 2,304 |
| 3 | H | R | D | L | R | A/S | A | 8 | 10,368 |
| 4 | H | R/K | D | L | A | T | R | 9 | 2,592 |
| 5 | H | R | D | L | A | A | R | 61 | 8,192 |
| 6 | H | K | D | L | A | A | R | 3 | 576 |
| 7 | H | R | D | I | A | A | R | 2 | 32 |
| Total | | | | | | | | 89 | 24,068 |

As a consequence of this division of the family into seven sub-sets, and as a further sequence of the fact that only known codons are taken into account when translating each of the variants of the signature motif into a consensus sequence, the total complexity of the library is significantly reduced. In the case of the family of tyrosine kinases, were an siRNA library to be produced without this division, the complexity of the library would be on the order of tens of millions of members. As can be seen from Table I, when such a division into the seven sub-sets listed in the table is done, the effect is to enable the production of a library having only 24,068 members. It will be appreciated that such a library is formed by combining all the DNA oligonucleotides synthesized on the basis of each of the seven consensus sequences and ligating these to the expression cassettes; in a preferred embodiment, in order to obtain a uniform complexity of 24,068 members, the seven batches of oligonucleotides are mixed together in direct proportion to their complexity prior to incorporation in the cassettes.

Utilizing any of the techniques described herein and in the Examples, it is possible to design efficient siRNA libraries comprising as little as 50 unique members or as many as one million or more members, although typically most libraries will be within the range of 20,000 to 100,000 unique members.

VII. Recombinant Vectors

The siRNA expression cassettes in accordance with the present invention may be incorporated in a vector that is capable of self-replication in host cells. As one of ordinary skill in the art would recognize, a large variety of such vectors may be suitable for use in connection with the present invention. Certain types of vectors allow the expression cassettes to be amplified. Other types of vectors are necessary for efficient introduction of the expression cassettes to cells and their stable expression once introduced. Any vector capable of accepting a DNA expression cassette of the present invention is contemplated as a suitable recombinant vector for the purposes of the invention. The vector may be any circular or linear length of DNA that either integrates into the host genome or is maintained in episomal form. Vectors may require additional manipulation or particular conditions to be efficiently inctroduced into a host cell (e.g., many expression plasmids), or can be part of a self-integrating, cell specific system, such as a recombinant virus.

Infection of cells with a viral vector is a preferred method for introducing the siRNA expression libraries of the present invention into cells. Exemplary mammalian viral vector systems include adenoviral vectors, adeno-associated type 1 (“AAV-1”) or adeno-associated type 2 (“AAV-2”) viral vectors, hepatitis delta vectors, live, attenuated delta viruses, herpes viral vectors, alphaviral vectors, or retroviral vectors (including lentiviral vectors).

The siRNA expression libraries in accordance with the invention may also be introduced into a host cell by transfection and other physical methods as is known in the art.

VIII. Uses for the Invention

One of the main applications of the present invention is the use of a library of siRNAs targeting a predetermined gene family for purposes of identifying genes involved in disease processes, utilizing techniques such as Inverse Genomics®. In general terms, these techniques involve transfecting or transducing a population of cells with the siRNA expression library and monitoring the population of cells for any phenotypic change, such as decrease or increase in expression of mRNA, proliferation, differentiation, apoptosis, or senescence, etc. For example, an siRNA library targeting the tyrosine kinase family can be used to identify tyrosine kinases that function in the normal apoptotic pathway as follows. The library is delivered to a population of cells by transduction with a retroviral vector. The transduced cells are then subjected to a stimulus that induces apoptosis in normal cells (e.g., treatment with etoposide, cisplatin, or ionizing radiation). The majority of the treated cells will die due to this treatment. However, if a tyrosine kinase participates in the apototic pathway downstream of the stimulus, then cells expressing an siRNA against this tyrosine kinase will survive due to the siRNA-mediated defect in the apoptotic pathway. SiRNA expression cassettes are rescued from the surviving cells by PCR or other methods known to those skilled in the art. Putative tyrosine kinases that function in the apoptotic pathway are then identified from the siRNA sequences.

The level of gene expression may also be determined at the protein level. Various immunological assays are routinely used by those skilled in the art to measure the level of a gene product, particularly using polyclonal or monoclonal antibodies that react specifically with a protein product. In addition, functional assays may also be performed to confirm the suppressed expression of one or more genes in transfected/transduced cells. Depending on the particular gene family and the known biological functions the gene products normally exert, specific assays can be designed for detecting decreased level of activity. For example, when the targeted gene family encodes enzymes, specific enzymatic assays can be carried out using suitable substrates to detect the enzymatic activity in the transfected or transduced cells. When the targeted genes encode kinases, for instance, the lack of kinase activity in transfected/transduced cells may be reflected in reduced level of phosphorylation of the substrates; when the targeted genes encode receptors, such as cytokine receptors, the diminished gene expression may be reflected in reduced response to the ligands; when the targeted genes encode tumor suppressors or oncogenes, the decreased gene expression may be reflected in changes, e.g., in the tumorigenic tendency and/or metastatic potential of the transfected or transduced cells. Other possible changes in phenotypes that may indicate the reduced gene expression include: viral susceptibility—HIV infection; autoimmunity—inactivation of lymphocytes; drug sensitivity—drug toxicity and efficacy; graft rejection—MHC antigen presentation, etc.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of nonessential parameters that could be changed or modified to yield essentially similar results.

### EXAMPLES

The symbols for amino acids used in the examples are as follows:

| | |
|---|---|
| A | Alanine |
| C | Cysteine |
| D | Aspartic acid |
| E | Glutamic acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |

### Example 1

Family of Human Nuclear Hormone Receptors (ZnF_C4 Domain)—45 Members

In this example, a single signature motif was designed based on the zinc finger domain present in all 45 known members of the nuclear hormone receptor family. A short segment of the zinc finger domain present in each of the 45 known family members is shown below. The consensus sequence was “reverse translated” utilizing only those codons that encode the signature motif region of known members of the family. Using a full 21-nucleotide consensus sequence to construct the siRNA library, the complexity would be 10,616,832. By reducing the length of the consensus sequence to 19 nucleotides, the complexity is reduced to 884,736. SiRNAs as short as 19 nucleotides are highly efficient at reducing their cognate mRNA levels [Czauderna, F. et al., Nucl. Acids Res. 31: 2705-2716 (2003)], therefore, reducing the length of the consensus sequence will have little, if any, effect on the degree of silencing produced by members of the library.

| | |
|---|---|
| tataatgcactgacctgtgaggggtgtaaaggtttcttcaggaga | (SEQ ID NO:1) |
| Y N A L T C E G C K G F F R R | (SEQ ID NO:2) |
| tacggcgtgcgcacctgtgagggctgcaaaggcttctttaagcgc | (SEQ ID NO:3) |
| Y G V R T C E G C K G F F K R | (SEQ ID NO:4) |
| tacggcgtgcgcacctgtgagggctgcaaaggcttctttaagcgc | (SEQ ID NO:5) |
| Y G V R T C E G C K G F F K R | (SEQ ID NO:6) |
| tacggcgtgcgaacctgcgagggctgcaagggctttttcaagaga | (SEQ ID NO:7) |
| Y G V R T C E G C K G F F K R | (SEQ ID NO:8) |
| tatggtgtccgcacatgtgagggctgcaagggcttcttcaagcgc | (SEQ ID NO:9) |
| Y G V R T C E G C K G F F K R | (SEQ ID NO:10) |
| tatggagcagtaacttgtgaaggctgcaaaggattttttaaaaga | (SEQ ID NO:11) |
| Y G A V T C E G C K G F F K R | (SEQ ID NO:12) |
| tacggggttatcacctgtgaggggtgcaagggcttcttccgccgg | (SEQ ID NO:13) |
| Y G V I T C E G C K G F F R R | (SEQ ID NO:14) |
| tacggagtcatcacatgtgaaggctgcaagggattctttaggagg | (SEQ ID NO:15) |
| Y G V I T C E G C K G F F R R | (SEQ ID NO:16) |
| tatggtgtcattacatgtgaaggctgcaagggctttttcaggaga | (SEQ ID NO:17) |
| Y G V I T C E G C K G F F R R | (SEQ ID NO:18) |
| tatggagtgtacagctgcgaggggtgcaagggcttcttcaagcgg | (SEQ ID NO:19) |
| Y G V Y S C E G C K G F F K R | (SEQ ID NO:20) |
| tacggggtttacagctgtgagggttgcaagggcttcttcaaacgc | (SEQ ID NO:21) |
| Y G V Y S C E G C K G F F K R | (SEQ ID NO:22) |
| tacggggtatacagttgtgaaggctgcaaagggttcttcaagagg | (SEQ ID NO:23) |
| Y G V Y S C E G C K G F F K R | (SEQ ID NO:24) |
| tacaacgtgctcagctgcgaaggctgcaagggcttcttccggcgc | (SEQ ID NO:25) |
| Y N V L S C E G C K G F F R R | (SEQ ID NO:26) |
| tacaatgttctgagctgcgagggctgcaagggattcttccgccgc | (SEQ ID NO:27) |
| Y N V L S C E G C K G F F R R | (SEQ ID NO:28) |
| tatgggatcatctcctgtgagggctgcaaagggtttttcaagcgg | (SEQ ID NO:29) |
| Y G I I S C E G C K G F F K R | (SEQ ID NO:30) |
| tatggggtcagctcttgtgaaggctgcaagggcttctttcgccga | (SEQ ID NO:31) |
| Y G V S S C E G C K G F F R R | (SEQ ID NO:32) |
| tatggggtcagctcttgtgaaggctgcaagggcttctttcgccga | (SEQ ID NO:33) |
| Y G V S S C E G C K G F F R R | (SEQ ID NO:34) |
| tatggggctgtcagttgtgaaggttgcaaaggtttcttcaaaagg | (SEQ ID NO:35) |
| Y G A V S C E G C K G F F K R | (SEQ ID NO:36) |
| tacggtgtcttcacctgcgagggctgcaagagctttttcaagcga | (SEQ ID NO:37) |
| Y G V F T C E G C K S F F K R | (SEQ ID NO:38) |
| tacggccagttcacgtgcgagggctgcaagagcttcttcaagcgc | (SEQ ID NO:39) |
| Y G Q F T C E G C K S F F K R | (SEQ ID NO:40) |
| tacggggtctacgcctgcgacggctgctcaggttttttcaaacgg | (SEQ ID NO:41) |
| Y G V Y A C D G C S G F F K R | (SEQ ID NO:42) |
| tatggcatctatgcctgcaacggctgcagcggcttcttcaagagg | (SEQ ID NO:43) |
| Y G I Y A C N G C S G F F K R | (SEQ ID NO:44) |
| tatggggcatccacctgtgatgggtgcaagggtttcttcagacgc | (SEQ ID NO:45) |
| Y G A S T C D G C K G F F R R | (SEQ ID NO:46) |
| tacggtgcctcgagctgtgacggctgcaagggcttcttccggagg | (SEQ ID NO:47) |
| Y G A S S C D G C K G F F R R | (SEQ ID NO:48) |
| tatggggtcagcgcctgtgagggctgcaagggcttcttccgccgc | (SEQ ID NO:49) |
| Y G V S A C E G C K G F F R R | (SEQ ID NO:50) |
| tatggggtcagcgcctgtgagggatgtaagggctttttccgcaga | (SEQ ID NO:51) |
| Y G V S A C E G C K G F F R R | (SEQ ID NO:52) |
| tacggtgtgcacgcctgcgagggctgcaagggctttttccgtcgg | (SEQ ID NO:53) |
| Y G V H A C E G C K G F F R R | (SEQ ID NO:54) |
| tatggagttcatgcttgcgaaggctgtaagggtttctttcggaga | (SEQ ID NO:55) |
| Y G V H A C E G C K G F F R R | (SEQ ID NO:56) |
| tacggtgttcatgcatgtgaggggtgcaagggcttcttccgtcgt | (SEQ ID NO:57) |
| Y G V H A C E G C K G F F R R | (SEQ ID NO:58) |
| tacggagtccacgcgtgtgaaggctgcaagggcttctttcggcga | (SEQ ID NO:59) |
| Y G V H A C E G C K G F F R R | (SEQ ID NO:60) |
| tatggagttcatgcttgtgaaggatgcaagggtttcttccggaga | (SEQ ID NO:61) |
| Y G V H A C E G C K G F F R R | (SEQ ID NO:62) |
| ttcaatgtcatgacatgtgaaggatgcaagggctttttcaggagg | (SEQ ID NO:63) |
| F N V M T C E G C K G F F R R | (SEQ ID NO:64) |
| tttaatgcgctgacttgtgagggctgcaagggtttcttcaggaga | (SEQ ID NO:65) |
| F N A L T C E G C K G F F R R | (SEQ ID NO:66) |
| taccgctgtatcacgtgtgaaggctgcaagggtttctttagaaga | (SEQ ID NO:67) |
| Y R C I T C E G C K G F F R R | (SEQ ID NO:68) |
| taccgctgtatcacttgtgagggctgcaagggcttctttcgccgc | (SEQ ID NO:69) |
| Y R C I T C E G C K G F F R R | (SEQ ID NO:70) |
| tacggactgctcacgtgtgagagctgcaagggcttcttcaagcgc | (SEQ ID NO:71) |
| Y G L L T C E S C K G F F K R | (SEQ ID NO:72) |
| tatgggctcctcacctgtgaaagctgcaagggattttttaagcga | (SEQ ID NO:73) |
| Y G L L T C E S C K G F F K R | (SEQ ID NO:74) |
| tatggggtagtcacctgtggcagctgcaaagttttcttcaaaaga | (SEQ ID NO:75) |
| Y G V V T C G S C K V F F K R | (SEQ ID NO:76) |
| tatggagctctcacatgtggaagctgcaaggtcttcttcaaaaga | (SEQ ID NO:77) |
| Y G A L T C G S C K V F F K R | (SEQ ID NO:78) |
| tatggtgtccttacctgtgggagctgtaaggtcttctttaagagg | (SEQ ID NO:79) |
| Y G V L T C G S C K V F F K R | (SEQ ID NO:80) |
| tatggagtcttaacttgtggaagctgtaaagttttcttcaaaaga | (SEQ ID NO:81) |
| Y G V L T C G S C K V F F K R | (SEQ ID NO:82) |
| tacggcgtggcctcctgcgaggcttgcaaggccttcttcaagagg | (SEQ ID NO:83) |
| Y G V A S C E A C K A F F K R | (SEQ ID NO:84) |
| tatggtgtggcatcctgtgaggcctgcaaagccttcttcaagagg | (SEQ ID NO:85) |
| Y G V A S C E A C K A F F K R | (SEQ ID NO:86) |
| tatggagtctggtcctgtgagggctgcaaggccttcttcaagaga | (SEQ ID NO:87) |
| Y G V W S C E G C K A F F K R | (SEQ ID NO:88) |
| tatggagtctggtcgtgtgaaggatgtaaggccttttttaaaaga | (SEQ ID NO:89) |
| Y G V W S C E G C K A F F K R | (SEQ ID NO:90) |

```
Signature Motif:
(T/S/A)-C-(D/E/G/N)-(G/S/A)-(C)-(K/S)-(A/G/S/V)
Consensus sequence (21 nt):
(A/T/G) (C/T) (A/G/T/C) TG(T/C) (A/G) (A/G) (A/C/G/T)
                T/S/A C D/E/G/N
(A/G) (C/G) (A/C/G/T) TG(T/C) (A/T) (A/C/G) (A/C/G)
              G/S/A C K/S
(A/G) (C/G/T) (A/C/G/T)
            G/S/V/A
Complexity: 2⁹ × 3⁴ × 4⁴ = 512 × 81 × 256 = 10,616,832 members
Consensus sequence (19 nt):
(A/T/G) (C/T) (A/G/T/C) TG(T/C) (A/G) (A/G) (A/C/G/T)
                T/S/A C D/E/G/N
(A/G) (C/G) (A/C/G/T) TG(T/C) (A/T) (A/C/G) (A/C/G)
              G/S/A C K/S
(A/G)--
G/S/V/A
Complexity: 2⁹ × 3³ × 4³ = 512 × 27 × 64 = 884,736 members
```

### Example 2

Family of Tyrosine Kinases—89 Members

This example shows the identification of seven variants of a portion of the catalytic domain of the family of tyrosine kinases. As shown in Table 1 above, these may then be used for the production of library of siRNAs targeting this domain having a reduced complexity of 24,068 unique members.

| Variant 1: 3 members | |
|---|---|
| gttcccatcatccaccgcgaccttaagtccagcaacatattgatcctc | (SEQ ID NO:91) |
| V P I I H R D L K S S N I L I L | (SEQ ID NO:92) |
| gtgcccatcctgcaccgggacctcaagtccagcaacattttgctactt | (SEQ ID NO:93) |
| V P I L H R D L K S S N I L L L | (SEQ ID NO:94) |
| gtgcccatcctgcaccgggacctcaagtccagcaacattttgctactt | (SEQ ID NO:95) |
| V P I L H R D L K S S N I L L L | (SEQ ID NO:96) |
| Signature Motif: | |
| H R D L K S S | |
| Consensus Sequence: | |
| CAC CG(C/G) GAC CT(C/T) AAG TCC AGC | |
| H R D L K S S | |
| Complexity: 2² = 4 members | |
| Variant 2: 3 members | |
| catggtatggtgcatagaaacctggctgcccgaaacgtgctactcaag | (SEQ ID NO:97) |
| H G M V H R N L A A R N V L L K | (SEQ ID NO:98) |
| aagaattgcatccaccgggacgtggcagcgcgtaacgtgctgttgacc | (SEQ ID NO:99) |
| K N C I H R D V A A R N V L L T | (SEQ ID NO:100) |
| atcaactgcgtgcacagggacattgctgtccggaacatcctggtggcc | (SEQ ID NO:101) |
| I N C V H R D I A V R N I L V A | (SEQ ID NO:102) |
| Signature Motif: | |
| H R D/N I/V/L A A/V R | |
| Consensus Sequence: | |
| CA(T/C) (C/A)G(G/A) (G/A)AC (A/C/G)T(T/G) GC(T/A) G(T/C) (C/G) | |
| H R D/N) (I/V/L) A (A/V) | |
| CG(A/T/G) | |
| R | |
| Complexity: 2⁸ × 3² = 256 × 9 = 2,304 members | |
| Variant 3: 8 members | |
| atgaactacgtccaccgggaccttcgtgcagccaacatcctggtggga | (SEQ ID NO:103) |
| M N Y V H R D L R A A N I L V G | (SEQ ID NO:104) |
| atgaactatattcaccgagatcttcgggctgctaatattcttgtagga | (SEQ ID NO:105) |
| M N Y I H R D L R A A N I L V G | (SEQ ID NO:106) |
| atgaattatatccatagagatctgcgatcagcaaacattctagtgggg | (SEQ ID NO:107) |
| M N Y I H R D L R S A N I L V G | (SEQ ID NO:108) |
| atgaactacattcaccgcgacctgagggcagccaacatcctggttggg | (SEQ ID NO:109) |
| M N Y I H R D L R A A N I L V G | (SEQ ID NO:110) |
| aagaattccatccaccgcgacctgcgggcggccaacatcctggtgtct | (SEQ ID NO:111) |
| M N S I H R D L R A A N I L V S | (SEQ ID NO:112) |
| aggaactacatccaccgagacctccgagctgccaacatcttggtctt | (SEQ ID NO:113) |
| R N Y I H R D L R A A N I L V S | (SEQ ID NO:114) |
| aagaactacattcaccgggacctgcgagcagctaatgttctggtctcc | (SEQ ID NO:115) |
| K N Y I H R D L R A A N V L V S | (SEQ ID NO:116) |
| cggaattatattcatcgtgaccttcgggctgccaacattctggtgtct | (SEQ ID NO:117) |
| R N Y I H R D L R A A N I L V S | (SEQ ID NO:118) |
| Signature Motif: | |
| H R D L R A/S A | |
| Consensus Sequence: | |
| CA(C/T) (C/A)G(A/C/G/T) GA(C/T) CT(C/G/T) (A/C)G(A/G/T) | |
| H R D L R | |
| (G/T)C(A/G/T) GC(A/C/T) | |
| (A/S) A | |
| Complexity: 2⁵ × 3⁴ × 4 = 32 × 81 × 4 = 10,368 members | |
| Variant 4: 9 Members | |
| ctgcattttgtgcaccgggacctggccacacgcaactgtctagtggg | (SEQ ID NO:119) |
| L H F V H R D L A T R N C L V G | (SEQ ID NO:120) |
| ctcaactttgtacatcgggacctggccacgcggaactgcctagttggg | (SEQ ID NO:121) |
| L N F V H R D L A T R N C L V G | (SEQ ID NO:122) |
| cttaattttgttcaccgagatctggccacacgaaactgtttagtgggt | (SEQ ID NO:123) |
| L N F V H R D L A T R N C L V G | (SEQ ID NO:124) |
| cgcgggctggtgcaccgagacctcgctacgcgcaacctactgctggcg | (SEQ ID NO:125) |
| R G L V H R D L A T R N L L L A | (SEQ ID NO:126) |
| aaaaggtatatccacagggatctggcaacgagaaatatattggtggag | (SEQ ID NO:127) |
| K R Y I H R D L A T R N I L V E | (SEQ ID NO:128) |
| cagcactttgtgcaccgagacctggccaccaggaactgcctggttgga | (SEQ ID NO:129) |
| Q H F V H R D L A T R N C L V G | (SEQ ID NO:130) |
| cagcacttcgtgcaccgcgatttggccaccaggaactgcctggtcggg | (SEQ ID NO:131) |
| Q H F V H R D L A T R N C L V G | (SEQ ID NO:132) |
| caccacgtggttcacaaggacctggccacccgcaatgtgctagtgtac | (SEQ ID NO:133) |
| H H V V H K D L A T R N V L V Y | (SEQ ID NO:134) |
| cgtaagtttgttcaccgagatttagccaccaggaactgcctggtgggc | (SEQ ID NO:135) |
| R K F V H R D L A T R N C L V G | (SEQ ID NO:136) |
| Signature Motif: | |
| H R/K D L A T R | |
| Consensus Sequence: | |
| CAC (A/C)(A/G)(A/C/G) GA(C/T) (C/T)T(A/C/G) GC(A/C/T) | |
| H R/K D L A | |
| AC(A/C/G) (A/C)G(A/C/G) | |
| T R | |
| Complexity: 2⁵ × 3⁵ = 32 × 81 = 2,592 members | |
| Variant 5: 61 members | |
| aagaagcttgtgcaccgcgacctggccgcccgcaacatcctggtctca | (SEQ ID NO:137) |
| K K L V H R D L A A R N I L V S | (SEQ ID NO:138) |
| aagaagcttgtgcaccgggacctagccgcccgcaacatcctggtctca | (SEQ ID NO:139) |
| K K L V H R D L A A R N I L V S | (SEQ ID NO:140) |
| aacaatttcgtgcatcgagacctggctgcccgcaatgtgctggtgtct | (SEQ ID NO:141) |
| N N F V H R D L A A R N V L V S | (SEQ ID NO:142) |
| cacgactacatccaccgagacctagccgcgcgcaacgtgctgctggac | (SEQ ID NO:143) |
| H D Y I H R D L A A R N V L L D | (SEQ ID NO:144) |
| cggcaatacgttcaccgggacttggcagcaagaaatgtccttgttgag | (SEQ ID NO:145) |
| R Q Y V H R D L A A R N V L V E | (SEQ ID NO:146) |
| cgtcgcttggtgcaccgcgacctggcagccaggaacgtactggtgaaa | (SEQ ID NO:147) |
| R R L V H R D L A A R N V L V K | (SEQ ID NO:148) |
| cggaacttcatccaccgagacctggctgctcggaattgcatgctggca | (SEQ ID NO:149) |
| R N F I H R D L A A R N C M L A | (SEQ ID NO:150) |
| aagaagtgcatacaccgagacctggcagccaggaatgtcctggtgaca | (SEQ ID NO:151) |
| K K C I H R D L A A R N V L V T | (SEQ ID NO:152) |
| caaaaatgtattcatcgagatttagcagccagaaatgttttggtaaca | (SEQ ID NO:153) |
| Q K C I H R D L A A R N V L V T | (SEQ ID NO:154) |
| cagaagtgcatccacagggacctggctgcccgcaatgtgctggtgacc | (SEQ ID NO:155) |
| Q K C I H R D L A A R N V L V T | (SEQ ID NO:156) |
| cagaagtgtattcacagagacttggctgccagaaacgtcctggtgacc | (SEQ ID NO:157) |
| Q K C I H R D L A A R N V L V T | (SEQ ID NO:158) |
| cggaagtgtatccaccgggacctggctgcccgcaatgtgctggtgact | (SEQ ID NO:159) |
| R K C I H R D L A A R N V L V T | (SEQ ID NO:160) |
| atgaagctcgttcatcgggacttggcagccagaaacatcctggtagct | (SEQ ID NO:161) |
| M K L V H R D L A A R N I L V A | (SEQ ID NO:162) |
| agaaagtgcattcatcgggacctggcagcgagaaacattcttttatct | (SEQ ID NO:163) |
| R K C I H R D L A A R N I L L S | (SEQ ID NO:164) |
| cgaaagtgcatccacagagacctggctgctcggaacattctgctgtcg | (SEQ ID NO:165) |
| R K C I H R D L A A R N I L L S | (SEQ ID NO:166) |
| cgaaagtgtatccacagggacctggcggcacgaaatatcctcttatcg | (SEQ ID NO:167) |
| R K C I H R D L A A R N I L L S | (SEQ ID NO:168) |
| aagaactgcgtccacagagacctggcggctaggaacgtgctcatctgt | (SEQ ID NO:169) |
| K N C V H R D L A A R N V L I C | (SEQ ID NO:170) |
| aaaaattgtgtccaccgtgatctggctgctcgcaacgtcctcctggca | (SEQ ID NO:171) |
| K N C V H R D L A A R N V L L A | (SEQ ID NO:172) |
| aagaattgtattcacagagacttggcagccagaaatatcctccttact | (SEQ ID NO:173) |
| K N C I H R D L A A R N I L L T | (SEQ ID NO:174) |
| aagtcgtgtgttcacagagacctggccgccaggaacgtgcttgtcacc | (SEQ ID NO:175) |
| K S C V H R D L A A R N V L V T | (SEQ ID NO:176) |
| aaacagtttattcacagggacctagctgccaggaacattttagttggt | (SEQ ID NO:177) |
| K Q F I H R D L A A R N I L V G | (SEQ ID NO:178) |
| aagcagttcatccacagggacctggctgcccggaatgtgctggtcgga | (SEQ ID NO:179) |
| K Q F I H R D L A A R N V L V G | (SEQ ID NO:180) |
| aagcagttcatccacagggacctggctgcccggaatgtgctggtcgga | (SEQ ID NO:181) |
| K Q F I H R D L A A R N V L V G | (SEQ ID NO:182) |
| atgaactatgtgcaccgtgacctggctgcccgcaacatcctcgtcaac | (SEQ ID NO:183) |
| M N Y V H R D L A A R N I L V N | (SEQ ID NO:184) |
| atgcatttcattcacagggatctggcagctagaaattgccttgtttcc | (SEQ ID NO:185) |
| M H F I H R D L A A R N C L V S | (SEQ ID NO:186) |
| aacaagtttgtgcaccgagatctagcagcccgcaactgcatggtgtcc | (SEQ ID NO:187) |
| N K F V H R D L A A R N C M V S | (SEQ ID NO:188) |
| aataagttcgtccacagagaccttgctgcccggaattgcatggtagcc | (SEQ ID NO:189) |
| N K F V H R D L A A R N C M V A | (SEQ ID NO:190) |
| aagaagtttgtgcatcgggacctggcagcgagaaactgcatggtcgcc | (SEQ ID NO:191) |
| K K F V H R D L A A R N C M V A | (SEQ ID NO:192) |
| aagagattcatacaccgggacctggcggccaggaactgcatgctgaat | (SEQ ID NO:193) |
| K R F I H R D L A A R N C M L N | (SEQ ID NO:194) |
| atgaactatgttcaccgtgacctggctgcccgcaacatcctcgtcaac | (SEQ ID NO:195) |
| M N Y V H R D L A A R N I L V N | (SEQ ID NO:196) |
| atgaactatgtgcaccgcgacctggctgctcgcaacatccttgtcaac | (SEQ ID NO:197) |
| M N Y V H R D L A A R N I L V N | (SEQ ID NO:198) |
| atgaattatgtgcatcgggacctggctgctaggaacattctggtcaac | (SEQ ID NO:199) |
| M N Y V H R D L A A R N I L V N | (SEQ ID NO:200) |
| atgggctatgtgcatagagatcttgctgccagaaacatcttaatcaac | (SEQ ID NO:201) |
| M G Y V H R D L A A R N I L I N | (SEQ ID NO:202) |
| cagaagtttgtgcacagggacctggctgcgcggaactgcatgctggac | (SEQ ID NO:203) |
| Q K F V H R D L A A R N C M L D | (SEQ ID NO:204) |
| aaaaagtttgtccacagagacttggctgcaagaaactgtatgctggat | (SEQ ID NO:205) |
| K K F V H R D L A A R N C M L D | (SEQ ID NO:206) |
| atgggctatgttcaccgagacctcgctgctcggaacatcttgatcaac | (SEQ ID NO:207) |
| M G Y V H R D L A A R N I L I N | (SEQ ID NO:208) |
| aggaattttcttcatcgagatttagctgctcgaaactgcatgttgcga | (SEQ ID NO:209) |
| R N F L H R D L A A R N C M L R | (SEQ ID NO:210) |
| aaaaactgtatacacagggaccttgctgcaagaaactgcctggtaggt | (SEQ ID NO:211) |
| K N C I H R D L A A R N C L V G | (SEQ ID NO:212) |
| aagtgctgcatccaccgggacctggctgctcggaactgcctggtgaca | (SEQ ID NO:213) |
| K C C I H R D L A A R N C L V T | (SEQ ID NO:214) |
| atgagctatgtgcatcgtgatctggccgcacggaacatcctggtgaac | (SEQ ID NO:215) |
| M S Y V H R D L A A R N I L V N | (SEQ ID NO:216) |
| atgagctacgtccaccgagacctggctgctcgcaacatcctagtcaac | (SEQ ID NO:217) |
| M S Y V H R D L A A R N I L V N | (SEQ ID NO:218) |
| atgggctatgttcaccgagacctcgctgctcggaacatcttgatcaac | (SEQ ID NO:219) |
| M G Y V H R D L A A R N I L I N | (SEQ ID NO:220) |
| atgggatatgttcacagggaccttgcagctcgcaatattcttgtcaac | (SEQ ID NO:221) |
| M G Y V H R D L A A R N I L V N | (SEQ ID NO:222) |
| cgtcgcttggtgcaccgcgacctggcagccaggaacgtactggtgaaa | (SEQ ID NO:223) |
| R R L V H R D L A A R N V L V K | (SEQ ID NO:224) |
| gtgcggctcgtacacagggacttggccgctcggaacgtgctggtcaag | (SEQ ID NO:225) |
| V R L V H R D L A A R N V L V K | (SEQ ID NO:226) |
| agacgactcgttcatcgggatttggcagcccgtaatgtcttagtgaaa | (SEQ ID NO:227) |
| R R L V H R D L A A R N V L V K | (SEQ ID NO:228) |
| aaaaacttcatccacagagatcttgctgcccgaaactgcctggtaggg | (SEQ ID NO:229) |
| K N F I H R D L A A R N C L V G | (SEQ ID NO:230) |
| aagcgctttattcaccgtgacctggctgcccgcaatctgctgttggct | (SEQ ID NO:231) |
| K R F I H R D L A A R N L L L A | (SEQ ID NO:232) |
| aagaactttgtgcaccgtgacctggcggcccgcaacgtcctgctggtt | (SEQ ID NO:233) |
| K N F V H R D L A A R N V L L V | (SEQ ID NO:234) |
| aagaactttgtgcaccgtgacctggcggcccgcaacgtcctgctggtt | (SEQ ID NO:235) |
| K N F V H R D L A A R N V L L V | (SEQ ID NO:236) |
| agcaattttgtgcacagagatctggctgcaagaaatgtgttgctagtt | (SEQ ID NO:237) |
| S N F V H R D L A A R N V L L V | (SEQ ID NO:238) |
| cagaattacatccaccgggacctggccgccaggaacatcctcgtcggg | (SEQ ID NO:239) |
| Q N Y I H R D L A A R N I L V G | (SEQ ID NO:240) |
| cagcgcgttgtgcaccgggacttggccgcccggaacgtgctcgtggac | (SEQ ID NO:241) |
| Q R V V H R D L A A R N V L V D | (SEQ ID NO:242) |
| cggaactacattcacagagatctggctgccagaaatgtcctcgttggt | (SEQ ID NO:243) |
| R N Y I H R D L A A R N V L V G | (SEQ ID NO:244) |
| aagaatttcatccatagagatcttgcagctcgtaactgcctagtggga | (SEQ ID NO:245) |
| K N F I H R D L A A R N C L V G | (SEQ ID NO:246) |
| aacagcttcatccacagagatctggctgccagaaattgtctagtaagt | (SEQ ID NO:247) |
| N S F I H R D L A A R N C L V S | (SEQ ID NO:248) |
| aatggctatattcatagggatttggcggcaaggaattgtttggtcagt | (SEQ ID NO:249) |
| N G Y I H R D L A A R N C L V S | (SEQ ID NO:250) |
| gcatgtgtcatccacagagacttggctgccagaaattgtttggtggga | (SEQ ID NO:251) |
| A C V I H R D L A A R N C L V G | (SEQ ID NO:252) |
| caccaattcatacaccgggacttggctgctcgtaactgcttggtggac | (SEQ ID NO:253) |
| H Q F I H R D L A A R N C L V D | (SEQ ID NO:254) |
| aagcagttccttcaccgagacctggcagctcgaaactgtttggtaaac | (SEQ ID NO:255) |
| K Q F L H R D L A A R N C L V N | (SEQ ID NO:256) |
| cacaattatgtccaccgggacctggctgccagaaacatcttggtgaat | (SEQ ID NO:257) |
| H N Y V H R D L A A R N I L V N | (SEQ ID NO:258) |
| Signature Motif: | |
| H R D L A A R | |
| Consensus Sequence: | |
| CA(C/T) (A/C)G(A/C/G/T) GA(C/T) (T/C)T(A/C/G/T) GC(A/C/G/T) | |
| H R D L A | |
| GC(A/C/G/T) (A/C)G(A/C/G/T) | |
| A R | |
| Complexity: 2⁴ × 4⁵ = 16 × 512 = 8,192 members | |
| Variant 6: 3 members | |
| agggaagtcatccacaaagacctggctgccaggaactgtgtcattgat | (SEQ ID NO:259) |
| R E V I H K D L A A R N C V I D | (SEQ ID NO:260) |
| aaccgctttgtgcataaggacttggctgcgcgtaactgcctggtcagt | (SEQ ID NO:261) |
| N R F V H K D L A A R N C L V S | (SEQ ID NO:262) |
| cacttctttgtccacaaggaccttgcagctcgcaatattttaatcgga | (SEQ ID NO:263) |
| H F F V H K D L A A R N I L I G | (SEQ ID NO:264) |
| Signature Motif: | |
| H K D L A A R | |
| Consensus Sequence: | |
| CA(C/T) AA(A/G) GAC(C/T)T(G/T) GC(A/T) GC(C/G/T) A/C)G(C/G/T) | |
| H K D L A A R | |
| Complexity: 2⁶ × 3² = 64 × 9 = 576 members | |
| Variant 7: 2 members | |
| aatcacttcatccacagggatattgccgcccggaactgcctgctgagc | (SEQ ID NO:265) |
| N H F I H R D I A A R N C L L S | (SEQ ID NO:266) |
| aaccacttcatccaccgagacattgctgccagaaactgcctcttgacc | (SEQ ID NO:267) |
| N H F I H R D I A A R N C L L T | (SEQ ID NO:268) |
| Signature Motif: | |
| H R D I A A R | |
| Consensus Sequence: | |
| CAC G(A/G) GA(C/T) ATT GC(C/T) GCC (A/C)G(A/G) | |
| H R D I A A R | |
| Complexity: 2⁵ = members | |

### Example 3

Family of Human Nuclear Hormone Receptors (ZnF_C4 Domain)—45 Members Divided Into 9 Groups

In this example, the 45 known members of the nuclear hormone receptor family are divided into 9 subgroups. The same segment of the Zinc Finger_C4 domain described in Example 1 was used to design individual signature motifs and consensus sequences for each of the 9 subgroups. As in Example 1, the consensus sequence was “reverse translated” utilizing only those codons that encode the signature motif region of known members of the subgroup. Division of the family into subgroups dramatically reduces the complexity from 10,616,832 (see Example 1) to 1,664.

| Variant 1: 9 members | |
|---|---|
| tataatgcactgacctgtgaggggtgtaaaggtttcttcaggaga | (SEQ ID NO:1) |
| Y N A L T C E G C K G F F R R | (SEQ ID NO:2) |
| tacggcgtgcgcacctgtgagggctgcaaaggcttctttaagcgc | (SEQ ID NO:3) |
| Y G V R T C E G C K G F F K R | (SEQ ID NO:4) |
| tacggcgtgcgcacctgtgagggctgcaaaggcttctttaagcgc | (SEQ ID NO:5) |
| Y G V R T C E G C K G F F K R | (SEQ ID NO:6) |
| tacggcgtgcgaacctgcgagggctgcaagggctttttcaagaga | (SEQ ID NO:7) |
| Y G V R T C E G C K G F F K R | (SEQ ID NO:8) |
| tatggtgtccgcacatgtgagggctgcaagggcttcttcaagcgc | (SEQ ID NO:9) |
| Y G V R T C E G C K G F F K R | (SEQ ID NO:10) |
| tatggagcagtaacttgtgaaggctgcaaaggattttttaaaaga | (SEQ ID NO:11) |
| Y G A V T C E G C K G F F K R | (SEQ ID NO:12) |
| tacggggttatcacctgtgaggggtgcaagggcttcttccgccgg | (SEQ ID NO:13) |
| Y G V I T C E G C K G F F R R | (SEQ ID NO:14) |
| tacggagtcatcacatgtgaaggctgcaagggattctttaggagg | (SEQ ID NO:15) |
| Y G V I T C E G C K G F F R R | (SEQ ID NO:16) |
| tatggtgtcattacatgtgaaggctgcaagggctttttcaggaga | (SEQ ID NO:17) |
| Y G V I T C E G C K G F F R R | (SEQ ID NO:18) |
| Signature Motif: | |
| T C E G C K G | |
| Consensus Sequence: | |
| A-C-(A/C/T) T-G-(C/T) G-A-(A/G) G-G-(C/G) T-G-(C/T) | |
| T C E G C | |
| A-A-(A/G) G-G-(A/C/T) | |
| K G | |
| Complexity: 2⁵ × 3² = 32 × 9 = 288 | |
| Variant 2: 9 members | |
| tatggagtgtacagctgcgaggggtgcaagggcttcttcaagcgg | (SEQ ID NO:19) |
| Y G V Y S C E G C K G F F K R | (SEQ ID NO:20) |
| tacggggtttacagctgtgagggttgcaagggcttcttcaaacgc | (SEQ ID NO:21) |
| Y G V Y S C E G C K G F F K R | (SEQ ID NO:22) |
| tacggggtatacagttgtgaaggctgcaaagggttcttcaagagg | (SEQ ID NO:23) |
| Y G V Y S C E G C K G F F K R | (SEQ ID NO:24) |
| tacaacgtgctcagctgcgaaggctgcaagggcttcttccggcgc | (SEQ ID NO:25) |
| Y N V L S C E G C K G F F R R | (SEQ ID NO:26) |
| tacaatgttctgagctgcgagggctgcaagggattcttccgccgc | (SEQ ID NO:27) |
| Y N V L S C E G C K G F F R R | (SEQ ID NO:28) |
| tatgggatcatctcctgtgagggctgcaaagggtttttcaagcgg | (SEQ ID NO:29) |
| Y G I I S C E G C K G F F K R | (SEQ ID NO:30) |
| tatggggtcagctcttgtgaaggctgcaagggcttctttcgccga | (SEQ ID NO:31) |
| Y G V S S C E G C K G F F R R | (SEQ ID NO:32) |
| tatggggtcagctcttgtgaaggctgcaagggcttctttcgccga | (SEQ ID NO:33) |
| Y G V S S C E G C K G F F R R | (SEQ ID NO:34) |
| tatggggctgtcagttgtgaaggttgcaaaggtttcttcaaaagg | (SEQ ID NO:35) |
| Y G A V S C E G C K G F F K R | (SEQ ID NO:36) |
| Signature Motif: | |
| S C E G C K G | |
| Consensus Sequence: | |
| (A/T)-(C/G)-(C/T) T-G-(C/T) G-A-(A/G) G-G-(C/G/T) T-G-C | |
| S C E G C | |
| A-A-(A/G) G-G-(A/C/G/T) | |
| K G | |
| Complexity: 2⁶ × 3 × 4 = 64 × 3 × 4 = 768 | |
| Variant 3: 2 members | |
| tacggtgtcttcacctgcgagggctgcaagagctttttcaagcga | (SEQ ID NO:37) |
| Y G V F T C E G C K S F F K R | (SEQ ID NO:38) |
| tacggccagttcacgtgcgagggctgcaagagcttcttcaagcgc | (SEQ ID NO:39) |
| Y G Q F T C E G C K S F F K R | (SEQ ID NO:40) |
| Signature Motif: | |
| T C E G C K S | |
| Consensus Sequence: | |
| A-C-(C/G) T-G-C G-A-G G-G-C T-G-C A-A-(A/G) A-G-(C/T) | |
| T C E G C K S | |
| Complexity: 2³ = 8 | |
| Variant 4: 2 members | |
| tacggggtctacgcctgcgacggctgctcaggttttttcaaacgg | (SEQ ID NO:41) |
| Y G V Y A C D G C S G F F K R | (SEQ ID NO:42) |
| tatggcatctatgcctgcaacggctgcagcggcttcttcaagagg | (SEQ ID NO:43) |
| Y G I Y A C N G C S G F F K R | (SEQ ID NO:44) |
| Signature Motif: | |
| A C D/N G C S G | |
| Consensus Sequence: | |
| G-C-C T-G-C (A/G)-A-C G-G-C T-G-C (A/T)-(C/G)-(A/C) | |
| A C D/N G C S | |
| G-G-(C/T) | |
| G | |
| Complexity: 2⁵ = 32 | |
| Variant 5: 2 members | |
| tatggggcatccacctgtgatgggtgcaagggtttcttcagacgc | (SEQ ID NO:45) |
| Y G A S T C D G C K G F F R R | (SEQ ID NO:46) |
| tacggtgcctcgagctgtgacggctgcaagggcttcttccggagg | (SEQ ID NO:47) |
| Y G A S S C D G C K G F F R R | (SEQ ID NO:48) |
| Signature Motif: | |
| T/S C D G C K G | |
| Consensus Sequence: | |
| A-(C/G)-C T-G-T G-A-(C/T) G-G-(C/G) T-G-C A-A-G | |
| S/T C D G C K | |
| G-G-(C/T) | |
| G | |
| Complexity: 2⁴ = 16 | |
| Variant 6: 7 members | |
| tatggggtcagcgcctgtgagggctgcaagggcttcttccgccgc | (SEQ ID NO:49) |
| Y G V S A C E G C K G F F R R | (SEQ ID NO:50) |
| tatggggtcagcgcctgtgagggatgtaagggctttttccgcaga | (SEQ ID NO:51) |
| Y G V S A C E G C K G F F R R | (SEQ ID NO:52) |
| tacggtgtgcacgcctgcgagggctgcaagggctttttccgtcgg | (SEQ ID NO:53) |
| Y G V H A C E G C K G F F R R | (SEQ ID NO:54) |
| tatggagttcatgcttgcgaaggctgtaagggtttctttcggaga | (SEQ ID NO:55) |
| Y G V H A C E G C K G F F R R | (SEQ ID NO:56) |
| tacggtgttcatgcatgtgaggggtgcaagggcttcttccgtcgt | (SEQ ID NO:57) |
| Y G V H A C E G C K G F F R R | (SEQ ID NO:58) |
| tacggagtccacgcgtgtgaaggctgcaagggcttctttcggcga | (SEQ ID NO:59) |
| Y G V H A C E G C K G F F R R | (SEQ ID NO:60) |
| tatggagttcatgcttgtgaaggatgcaagggtttcttccggaga | (SEQ ID NO:61) |
| Y G V H A C E G C K G F F R R | (SEQ ID NO:62) |
| Signature Motif: | |
| A C E G C K G | |
| Consensus Sequence: | |
| G-C-(A/C/G/T) T-G-(C/T) G-A-(A/G) G-G-(A/C/G) T-G-(C/T) | |
| A C E G C | |
| A-A-G G-G-(C/T) | |
| K G | |
| Complexity: 2⁴ × 3 × 4 = 16 × 12 = 192 | |
| Variant 7: 6 members | |
| ttcaatgtcatgacatgtgaaggatgcaagggctttttcaggagg | (SEQ ID NO:63) |
| F N V M T C E G C K G F F R R | (SEQ ID NO:64) |
| tttaatgcgctgacttgtgagggctgcaagggtttcttcaggaga | (SEQ ID NO:65) |
| F N A L T C E G C K G F F R R | (SEQ ID NO:66) |
| taccgctgtatcacgtgtgaaggctgcaagggtttctttagaaga | (SEQ ID NO:67) |
| Y R C I T C E G C K G F F R R | (SEQ ID NO:68) |
| taccgctgtatcacttgtgagggctgcaagggcttctttcgccgc | (SEQ ID NO:69) |
| Y R C I T C E G C K G F F R R | (SEQ ID NO:70) |
| tacggactgctcacgtgtgagagctgcaagggcttcttcaagcgc | (SEQ ID NO:71) |
| Y G L L T C E S C K G F F K R | (SEQ ID NO:72) |
| tatgggctcctcacctgtgaaagctgcaagggattttttaagcga | (SEQ ID NO:73) |
| Y G L L T C E S C K G F F K R | (SEQ ID NO:74) |
| Signature Motif: | |
| T C E G/S C K G | |
| Consensus Sequence: | |
| A-C-(A/C/G/T) T-G-T G-A-(A/G) (A/G)-G-(A/C) T-G-C A-A-G | |
| T C E G/S C K | |
| G-G(A/C/T) | |
| G | |
| Complexity: 2³ × 3 × 4 = 8 × 3 × 4 = 96 | |
| Variant 8: 4 members | |
| tatggggtagtcacctgtggcagctgcaaagttttcttcaaaaga | (SEQ ID NO:75) |
| Y G V V T C G S C K V F F K R | (SEQ ID NO:76) |
| tatggagctctcacatgtggaagctgcaaggtcttcttcaaaaga | (SEQ ID NO:77) |
| Y G A L T C G S C K V F F K R | (SEQ ID NO:78) |
| tatggtgtccttacctgtgggagctgtaaggtcttctttaagagg | (SEQ ID NO:79) |
| Y G V L T C G S C K V F F K R | (SEQ ID NO:80) |
| tatggagtcttaacttgtggaagctgtaaagttttcttcaaaaga | (SEQ ID NO:81) |
| Y G V L T C G S C K V F F K R | (SEQ ID NO:82) |
| Signature Motif: | |
| T C G S C K V | |
| Consensus Sequence: | |
| A-C-(A/C/T) T-G-T G-G-(A/C/G) A-G-C T-G-(C/T) A-A-(A/G) | |
| T C G S C K | |
| G-T-(C/T) | |
| V | |
| Complexity: 2³ × 3² = 8 × 9 = 72 | |
| Variant 9: 4 members | |
| tacggcgtggcctcctgcgaggcttgcaaggccttcttcaagagg | (SEQ ID NO:83) |
| Y G V A S C E A C K A F F K R | (SEQ ID NO:84) |
| tatggtgtggcatcctgtgaggcctgcaaagccttcttcaagagg | (SEQ ID NO:85) |
| Y G V A S C E A C K A F F K R | (SEQ ID NO:86) |
| tatggagtctggtcctgtgagggctgcaaggccttcttcaagaga | (SEQ ID NO:87) |
| Y G V W S C E G C K A F F K R | (SEQ ID NO:88) |
| tatggagtctggtcgtgtgaaggatgtaaggccttttttaaaaga | (SEQ ID NO:89) |
| Y G V W S C E G C K A F F K R | (SEQ ID NO:90) |
| Signature Motif: | |
| S C E A/G C K A | |
| Consensus Sequence: | |

```
T-C-(C/G) T-G-(C/T) G-A-(A/G) G-(C/G)-(A/C/T) T-G-(C/T)
        S C E A/G C
A-A-(A/G) G-C-C
        K A
Complexity: 2⁶ × 3 = 64 × 3 = 192
Total Complexity of library: the sum of the complexities
of subgroups 1-9 = 1,664.
The library is constructed from the following semi-randomized
oligonucleotides:
Variant 1 (SEQ ID NO:269)
5′-pCCAGGACGACAAAAAGACHTGYGARGGSTGYAARGGHCTTTTTAGGCTTTTCGG-3′
Variant 2 (SEQ ID NO:270)
5′-pCCAGGACGACAAAAAGWSYTGYGARGGBTGCAARGGNCTTTTTAGGCTTTTCGG-3′
Variant 3 (SEQ ID NO:271)
5′-pCCAGGACGACAAAAAGACSTGCGAGGGCTGCAARAGYCTTTTTAGGCTTTTCGG-3′
Variant 4 (SEQ ID NO:272)
5′-pCCAGGACGACAAAAAGCCTGCRACGGCTGCWSMGGYCTTTTTAGGCTTTTCGG-3′
Variant 5 (SEQ ID NO:273)
5′-pCCAGGACGACAAAAAGASCTGTGAYGGSTGCAAGGGYCTTTTTAGGCTTTTCGG-3′
Variant 6 (SEQ ID NO:274)
5′-pCCAGGACGACAAAAAGCNTGYGARGGVTGYAAGGGYCTTTTTAGGCTTTTCGG-3′
Variant 7 (SEQ ID NO:275)
5′-pCCAGGACGACAAAAAGACNTGTGARRGMTGCAAGGGHCTTTTTAGGCTTTTCGG-3′
Variant 8 (SEQ ID NO:276)
5′-pCCAGGACGACAAAAAGACHTGTGGVAGCTGYAARGTYCTTTTTAGGCTTTTCGG-3′
Variant 9 (SEQ ID NO:277)
5′-pCCAGGACGACAAAAAGTCSTGYGARGSHTGYAARGCCTTTTTAGGCTTTTCGG-3′
```

In the above, mixtures of nucleotides (wobbles) are denoted using the following standard nomenclature:

TABLE 2

| Wobble | Nucleotides |
|---|---|
| B | C + G + T |
| D | A + G + T |
| H | A + C + T |
| K | G + T |
| M | A + C |
| N | A + C + G + T |
| R | A + G |
| S | C + G |
| V | A + C + G |
| W | A + T |
| Y | C + T |

The semi-randomized oligonucleotides are resuspended in TE buffer and combined in direct proportion to their complexities to a final concentration of 0.92 μM. One hundred eight pmol of the semi-randomized oligonucleotide mixture is combined with 21.6 pmol each of adapter oligonucleotides Univ-1 (FseI) and Univ-2(AscI).

| | |
|---|---|
| Univ-1(FseI): | |
| 5′-CTTTTTGTCGTCCTGGCCGG-3′ | (SEQ ID NO:278) |
| Univ-2(AscI): | |
| 5′-pCGCGCCGAAAAGCCTAAAAAG-3′ | (SEQ ID NO:279) |

The oligonucleotides are annealed by heating to 70° C. for 5 minutes and slowly cooling to room temperature (˜3 hours). The annealed oligonucleotides are ligated to 0.216 pmol of an FseI/AscI-digested vector bearing opposing human U6 and murine U6 promoters. Construction of this vector is described in U.S. patent application Ser. No. 10/626,512. The nucleotide sequence of the human U6 and murine U6 promoters between the TATA box and the transcription start site was modified to contain FseI and AscI restriction sites, respectively, as indicated below:

Human U6/Murine U6 Opposing Promoter Cassette

(FseI and AscI Sites in Lower Case Letters):

| | |
|---|---|
| GGATCCAAGCTTAAGGTCGGGCAGGAAGAGGGCC | (SEQ ID NO:280) |
| TATTTCCCATGATTCCTTCATATTTGCATATACG | |
| ATACAAGGCTGTTAGAGAGATAATTAGAATTAAT | |
| TTGACTGTAAACACAAAGATATTAGTACAAAATA | |
| CGTGACGTAGAAAGTAATAATTTCTTGGGTAGTT | |
| TGCAGTTTTAAAATTATGTTTTAAAATGGACTAT | |
| CATATGCTTACCGTAACTTGAAAGTATTTCGATT | |
| TCTTGGCTTTATATATCggccggccTCGAggcgc | |
| gccATATTTATAGTCTCAAAACACACAATTACTT | |
| TACAGTTAGGGTGAGTTTCCTTTTGTGCTGTTTT | |
| TTAAAATAATAATTTAGTATTTGTATCTCTTATA | |
| GAAATCCAAGCCTATCATGTAAAATGTAGCTAGT | |
| ATTAAAAAGAACAGATTATCTGTCTTTTATCGCA | |
| CATTAAGCCTCTATAGTTACTAGGAAATATTATA | |
| TGCAAATTAACCGGGGCAGGGGAGTAGCCGAGCT | |
| TCTCCCACAAGTCTGTGCGAGGGGGCCGGCGCGG | |
| GCCTAGAGATGGCGGCGTCGGATCC | |

Ligation is performed overnight at 16° C. One-fifth of the ligation reaction is used to transform electrocompetent bacteria (DH12S), resulting in 10⁶-10⁷ cfu/μg DNA.

The relatively low complexity (1,664) permits the delivery of the resulting library to the host cells by transient transfection in a 96-well format. The library is arrayed by picking ˜4,000 individual colonies and inoculating 750 μl/well of TB media (containing appropriate antibiotics) in 2-ml deep well 96-well plates (VWR). Following incubation for 20 hours, the cultures are pooled in groups of 10. DNA minipreps (Qiaprep Spin Miniprep Kits, Qiagen) are prepared from 1.5 ml of pooled bacterial culture. (The remainder of each culture is aliquotted and frozen for future use.) The purified DNA from each pool is quantitated using Rediplate 96 PicoGreen dsDNA Quantitation Kits (Molecular Probes). DNA from each pool is diluted to 100 ng/μl and stored in 96-well plates. Each well contains DNA encoding up to 10 unique siRNAs. Transfection of target cells is performed in a 96-well format using standard methods.

1. A method for generating an siRNA expression library for selective post-transcriptional silencing of genes encoding a family of proteins, the method comprising:
i. identifying a consensus sequence for the family of proteins; and,
ii. generating an siRNA expression library whose members encode siRNA molecules that target at least all mRNA encoding all known members of the family of proteins.

2. The method of claim 1, wherein the consensus sequence comprises between 15 to 30 nucleotides.

3. The method of claim 1, wherein the consensus sequence comprises between 18 to 24 nucleotides.

4. The method of claim 1, wherein the library comprises between 50 and one million unique members.

5. The method of claim 1, wherein the library comprises between 20,000 and 100,000 unique members.

6. The method of claim 1, wherein the family of proteins is selected from the group consisting of: G protein coupled receptors, ion channels, receptor tyrosine kinases, non-receptor tyrosine kinases, nuclear hormone receptors, GTPases, ATPases, serine/threonine kinases, proteases, matrix metalloproteinases (MMPs), GTPase-activating proteins (GAPs) and E3 ubiquitin ligases.

7. The method according to claim 1 wherein the step of identifying a consensus sequence comprises identifying at least one signature motif for the family of proteins.

8. The method according to claim 1 wherein the step of identifying a consensus sequence comprises identifying two or more variants of a signature motif for the family of proteins.

9. An siRNA expression library for selective post-transcriptional silencing of genes encoding a family of proteins, wherein members of the library encode siRNA molecules that are of between 15 to 30 nucleotides in length and target at least all mRNA encoding all known members of the family of proteins, and wherein the library comprises up to one million unique members.

10. The library of claim 9, wherein the library comprises up to 100,000 unique members.

11. The library of claim 9, wherein the family of proteins is selected from the group consisting of: G protein coupled receptors, ion channels, receptor tyrosine kinases, non-receptor tyrosine kinases, nuclear hormone receptors, GTPases, ATPases, serine/threonine kinases, proteases, matrix metalloproteinases (MMPs), GTPase-activating proteins (GAPs) and E3 ubiquitin ligases.

12. The library of claim 9, wherein the siRNA molecules are between 18 to 24 nucleotides in length.
